SEQUENCE LISTING (7 sequences)

SEQ ID NO: 1 - protein, 31 aa, Homo sapiens
HAEGTFTSDVSSYLEGQAAKEFIAWLVKGRG

SEQ ID NO: 2 - protein, 30 aa, Homo sapiens
  MOD_RES 30: note = Amidated
HAEGTFTSDVSSYLEGQAAKEFIAWLVKGR

SEQ ID NO: 3 - protein, 42 aa, Homo sapiens
YAEGTFISDYSIAMDKIHQQDFVNWLLAQKGKKNDWKHNITQ

SEQ ID NO: 4 - protein, 30 aa, Homo sapiens
  MOD_RES 30: note = Amidated
YAEGTFISDYSIAMDKIHQQDFVNWLLAQK

SEQ ID NO: 5 - protein, 38 aa, synthetic construct
  MOD_RES 20: note = Side chain modified with (2-[2-(2-Amino-ethoxy)- ethoxy]-acetyl)2-(Gamma-Glu)1-CO-(CH2)18-C02H
  MOD_RES 38: note = Amidated
YUEGTGTSDYSIULDKIAQKAFVQWLIAGGPSSGAPPS

SEQ ID NO: 6 - protein, 31 aa, synthetic construct
  MOD_RES 10: note = Side chain modified to -(CH2)4-NH-C(=O)-(CH2)2-C(CH3)(p-C6H5-OH)2
  MOD_RES 31: note = Amidated
HAEGTFTSDKSSYLEGQAAKEFIAWLVKGRG

SEQ ID NO: 7 - protein, 30 aa, synthetic construct
  MOD_RES 13: note = Side chain modified to -(CH2)4-NH-C(=O)-(CH2)2-C(CH3)(p-C6H5-OH)2
  MOD_RES 30: note = Amidated
YAEGTFISDYSIKMDKIHQQDFVNWLLAQK

(19) United States
(12) Patent Application Publication
(10) Pub. No.: US 2025/0026782 A1
(43) Pub. Date: Jan. 23, 2025

(54) ACTIVATING APPENDAGES TO INDUCE POLYPHARMACOLOGY IN PEPTIDE HORMONE ANALOGUES, INCLUDING DUAL GLP-1R / GIP AGONISM

(71) Applicant: Wisconsin Alumni Research Foundation, Madison, WI (US)

(72) Inventors: Samuel Helmer Gellman, Madison, WI (US); Rylie Kathleen Morris, Madison, WI (US); Kyle Alexander Brown, Queens, NY (US)

(73) Assignee: Wisconsin Alumni Research Foundation, Madison, WI (US)

(21) Appl. No.: 18/752,309

(22) Filed: Jun. 24, 2024

Related U.S. Application Data
(60) Provisional application No. 63/522,925, filed on Jun. 23, 2023.

(51) Int. Cl. C07K 1/00; A61K 38/00; C07K 14/605

(52) U.S. Cl. CPC C07K 1/006; C07K 14/605; A61K 38/00

(57) ABSTRACT

Compounds, method of making the compounds, and methods of using the compounds to treat Type 2 diabetes and obesity in mammals, including humans. The compounds are dual GLP-1 receptor and GIP receptor agonists in which at least one side chain of a residue in GLP-1 and/or GIP is modified such that the modified GLP-1 and GIP are agonists of both GLP-1 receptor and GIP receptor.

### CROSS-REFERENCE TO RELATED APPLICATIONS

Priority is hereby claimed to provisional application Ser. No. 63/522,925, filed Jun. 23, 2023, which is incorporated herein by reference.

### FEDERAL FUNDING STATEMENT

This invention was made with government support under GM056414 awarded by the National Institutes of Health. The government has certain rights in the invention.

### SEQUENCE LISTING

The instant application contains a Sequence Listing which has been submitted in an XML file with the USPTO through Patent Center and is hereby incorporated by reference in its entirety. The Sequence Listing XML, created on Sep. 17, 2024, is named “REPLACEMENT SEQ LIST--09824539-P230413US02.xml” and is 10,464 bytes in size.

### BACKGROUND

Tirzepatide is the first dual-action GIP/GLP-1 receptor agonist to reach the market in the United States. It is an agonist of both the glucose-dependent insulinotropic polypeptide (GIP) receptors and the glucagon-like peptide-1 (GLP-1) receptors. In the United States, it is approved for use as a once-weekly injection to treat Type-2 diabetes. It is sold under the trademark “MOUNJARO®” (a registered trademark of Eli Lilly and Company, Indianapolis, Indiana). See Bokvist et al., U.S. Pat. No. 9,474,780, issued Jun. 25, 2016 (which discloses and claims the tirzepatide molecule). See also Corvari et al, U.S. Pat. No. 11,357,820, issued Jun. 14, 2022, Benson et al. US 2021/0338781, published Nov. 4, 2021, and Benson et al. US 2020/0023040, published Jan. 23, 2020.

GIP and GLP-1 are peptide hormones relevant to the onset of diabetes. Mature human GIP is a gastrointestinal regulatory peptide that plays a physiological role in glucose homeostasis by stimulating insulin secretion from pancreatic beta cells in the presence of glucose. Mature human GLP-1 is a peptide that stimulates insulin secretion, and inhibits glucagon secretion, gastric emptying, and food intake. GIP and GLP-1 are known as “incretins.” In the human body, GLP-1 and GIP are rapidly inactivated by the enzyme dipeptidyl peptidase-4.

Despite insulin being available as an injectable treatment for diabetes since the 1920's, diabetes continues to be a chronic public health issue. Thus, intense research continues to find alternative treatments for diabetes and related metabolic disorders. In recent years, considerable research effort has been focused on incorporating (GLP-1) into a viable treatment for diabetes. GLP-1 is secreted by ileal L cells. Secretion is dependent upon the presence of nutrients in the lumen of the small intestine. GLP-1 is a potent anti-hyperglycemic hormone. Additionally, GLP-1 is known to inhibit pancreatic β-cell apoptosis and to stimulate the proliferation and differentiation of insulin-secreting β-cells. It is secreted as a pro-protein, which is then post-translationally modified to yield two physiologically active forms: GLP-1 (7-37) (SEQ ID NO: 1) and GLP-1 (7-36)-NH₂ (SEQ ID NO: 2). See, for example, Gellman et al. U.S. Pat. No. 10,723,779, issued Jul. 28, 2020.

GLP-1 (7-36)-NH₂ is a polypeptide having 30 amino acid residues (residues 7-36 of the proglucagon precursor), with a primary amide (NH₂) bonded to the carboxy terminus. GLP-1 (7-37) is a polypeptide having 31 amino acid residues (residues 7-37 of the proglucagon precursor). Both versions have the same insulinotropic hormone secretion action. For a discussion of GLP-1 and the functionally related insulinotropic hormones extendin-3 and extendin-4, see e.g., U.S. Pat. No. 5,424,286, issued Jun. 13, 1995.

GLP-1 is the natural agonist for GLP-1R, a G protein-coupled receptor (GPCR) that is displayed on the surface of pancreatic β cells. Activation of GLP-1R augments glucose-dependent insulin release from β cells and, as noted above, promotes β cell survival. These properties are attractive for treatment of Type 2 diabetes. However, GLP-1 is rapidly degraded by peptidases in vivo. Its half-life in vivo is less than two (2) minutes. Efforts to develop small-molecule agonists of GLP-1R have not been successful, presumably because receptor activation requires contact over an extended surface. All non-natural GLP-1R agonists reported to date consist exclusively of α-amino acid residues. In the non-natural GLP-1R agonists now known, in vivo activity is prolonged via several approaches, such as varying the sequence of α-amino acid residues, incorporating stabilizing appendages, and/or utilizing specialized delivery strategies. GLP-1 derivatives have been approved for sale for use in humans in the United States. See, for example, Victoza®-brand liraglutide (rDNA origin) for injection, marketed commercially by Novo Nordisk, Inc., Plainsboro, N.J. See also U.S. Pat. Nos. 6,268,343; 6,458,924; 7,235,627; and 8,114,833.

GIP is an incretin hormone secreted by the enteroendocrine K cells in response to nutrient intake The primary biologically active form of GIP is GIP (1-42) (SEQ ID NO: 3), which consists of 42 amino acids and is the full-length, native form of the peptide. Upon release into the bloodstream, GIP (1-42) binds to GIPR on pancreatic beta cells, stimulating insulin secretion in a glucose-dependent manner. Additionally, amidated truncated forms of GIP, such as GIP (1-30)-NH₂ (SEQ ID NO: 4), have been identified as biologically active variants. GIP (1-30)-NH₂ retains the insulinotropic activity of the full-length peptide but consists of only the first 30 amino acids of GIP with an added amide group at the C-terminus.

The term “diabetes mellitus” or simply “diabetes” is used herein in a very broad sense to encompass metabolic disorders in which a subject has high blood sugar (i.e., hyperglycemia). Hyperglycemic conditions have various etiologies, such as because the pancreas does not produce enough insulin, or because cells do not respond to the insulin that is produced. There are several recognized sub-types of diabetes, some of which are better understood than others. Type 1 diabetes is characterized by the complete failure of the body to produce insulin or the failure of the body to produce enough insulin. Type 2 diabetes generally results from insulin resistance, a condition in which cells fail to use insulin properly. Type 2 diabetes sometimes co-presents with an insulin deficiency. Gestational diabetes occurs when pregnant women without a previous diagnosis of diabetes develop hyperglycemia. Less common forms of diabetes include congenital diabetes (due to genetic defects relating to insulin secretion), cystic fibrosis-related diabetes, steroid diabetes induced by high doses of glucocorticoids, and several forms of monogenic diabetes (also known as maturity onset diabetes of the young). These last two terms are catch-all phrases that refer to several hereditary forms of diabetes caused by mutations in a single, autosomal dominant gene (as contrasted to more complex, polygenic etiologies resulting in hyperglycemia).

Insofar as GIP and GLP-1 play roles in the secretion of insulin and the inhibition of glucagon, the proteins and their respective receptors are directly implicated in the onset and progression of Type 2 diabetes. In Type 2 diabetes mellitus, the combined effects of impaired insulin secretion and insulin resistance are associated with chronically elevated blood glucose levels. Left untreated, Type 2 diabetes leads to a host of diverse and dire complications, including heart disease, kidney damage, peripheral neuropathy (nerve pain), and diabetic retinopathy that can lead to vision loss. Despite more than 100 years having passed since the discovery of insulin by Banting and Best, diabetes remains a worldwide public health concern. Thus, improved methods to treat diabetes remains a long-felt and unmet need.

### SUMMARY

Disclosed herein is a method of making dual GLP-1 receptor and GIP receptor agonists. The method comprising modifying at least one side chain of a residue in GLP-1 and/or GIP, to yield a modified GLP-1 and/or GIP, wherein the modified GLP-1 and GIP are agonists of both GLP-1 receptor and GIP receptor. Also disclosed herein are the resulting compounds, along with pharmaceutical compositions containing the compounds for use (primarily) to treat Type 2 diabetes and also (secondarily) to treat obesity, along with other diseases.

In a specific version of the method and the corresponding compounds, at least one side chain of a residue in GLP-1 and/or GIP is modified to contain a side chain comprising:

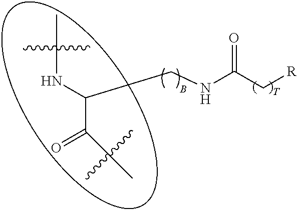

wherein:
the circled portion is the backbone of the GLP-1 or GIP peptide (or fragment thereof, as defined herein);
B is an integer of from 1 to 6;
T is an integer of from 0 to 6, or the carbon atom bearing the subscript T is a substituted or unsubstituted C₃-C₆-cycloalkyl and T=1; and
R is selected from the group consisting of polycyclic aromatic hydrocarbons and

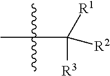

wherein R¹ is independently selected from the group consisting of an unsubstituted polycyclic aromatic hydrocarbon, a polycyclic aromatic hydrocarbon having up to three substitutions, an unsubstituted heterocycle, a heterocycle having up to three substitutions, and

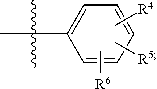

wherein R⁴, R⁵, and R⁶ are independently selected from the group consisting of H, halogen, —OH, C₁-C₆-linear or branched, unsubstituted or alkyl, and —O—C₁-C₆-linear or branched, substituted or unsubstituted alkyl; and
R² and R³ are independently selected from the group consisting of H, —OH, —C₁-C₆-linear or branched, substituted or unsubstituted alkyl, —C₁-C₆-linear or branched, substituted or unsubstituted alkylaryl, —O—C₁-C₆-linear or branched, substituted or unsubstituted alkyl, —O—C₁-C₆-linear or branched, substituted or unsubstituted alkylaryl, and the groups listed for R¹; and salts thereof.

One or more side chains in a residue of the GLP-1 and/or GIP may also be modified to contain a “fatty acid-like” side chain (as that term is defined herein).

In one version of the method, one (1) and only (1) side chain of a residue in the GLP-1 and/or GIP is modified.

In certain versions of the method, compounds, and compositions, R is

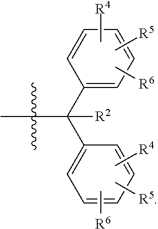

Here, R², R⁴, R⁵, and R⁶ are as defined above, or independently selected from the group consisting of H, —OH, —CH₃, —O—CH₃, and halogen. In still other versions of the disclosure, “T” and “B” are integers of from 1 to 4, R⁴ R⁵, and R⁶ are independently selected form the group consisting of —OH, —O—CH₃, and halogen, and R² is H, halogen, or —CH₃.

In a different version, the carbon atom bearing the subscript T is cyclohexyl and T=1. Also disclosed herein are methods of treating diabetes and/or obesity in a mammal. The method comprises administering an anti-diabetes-effective amount or a weight loss-effective of a compound or composition of matter as disclosed herein to a mammal, including humans.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1B show that GLP-1 and GIP have no cross-reactivity at their cognate receptors. FIG. 1A shows that GIP (1-30) (SEQ ID NO: 4) does not activate the GLP-1 receptor.

FIG. 1B shows that GLP-1 (SEQ ID NO: 1) does not activate the GIP receptor.

FIGS. 2A-2B show activation of the GLP-1 receptor and the GIP receptor by tirzepatide (SEQ ID NO: 5). FIG. 2A shows activation of the GLP-1 receptor by GLP-1 itself and tirzepatide. As in FIG. 1A, GIP has no effect on the GLP-1 receptor. FIG. 2B shows activation of the GIP receptor by GIP itself and tirzepatide. As in FIG. 1B, GLP-1 has no effect on the GIP receptor.

FIG. 3 shows that a single side chain modification can turn GLP-1 into a dual GLP-1 receptor and GIP receptor agonist. The upper portion of FIG. 3 shows GLP-1* (SEQ ID NO: 6), a GLP-1 peptide in which the side chain of the lysine at position 16 has been modified. (Note: the native residue at this position in GLP-1 is designated Val-16, because the numbering of residues in mature GLP-1 conventionally begins with His-7.) The lower portion of FIG. 3 shows that GLP-1* acts as an agonist for both the GLP-1 receptor (lower left) and the GIP receptor (lower right).

FIG. 4 shows that a single side chain modification can turn GIP into a dual GIP receptor and GLP-1 receptor agonist. The upper portion of FIG. 4 shows GIP* (SEQ ID NO: 7), a GIP peptide in which the side chain of the lysine at position 10 has been modified. The native residue at this position in GIP is Tyr-10. The lower portion of FIG. 4 shows that GIP* acts as an agonist for both the GLP-1 receptor (lower left) and the GIP receptor (lower right).

FIG. 5 shows that the GIP receptor agonist activity of GLP-1* is sensitive to the length of the side chain modification. The results show that the C4-length side chain has greatest GIP receptor agonist activity compared to the C1-, C2-, and C3-length.

FIG. 6 shows activation of GIPR by GLP-1 analogues with side chain modification at various backbone positions (12, 16, 18, 22, 26). This figure shows the production of cAMP by the cells. The scatter plots depict the dose-response relationship between the logarithm of peptide concentration and GIPR activation. The corresponding EC₅₀ values are shown in the table below the plots.

FIGS. 7 and 8 show activation of GLP-1R by GLP-1 analogues with side chain modification at backbone position 16. FIG. 7 presents the results from the CAMP production assay. FIG. 8 presents the results from the β-arrestin-2 recruitment assay. The scatter plots depict the dose-response relationship between the logarithm of peptide concentration and GLP-1R activation. The corresponding EC₅₀ values are shown in the table below the plots.

FIG. 9 shows activation of GIPR by GLP-1 analogues with varying length of the side chain modification at backbone position 16, revealed by cAMP production. The scatter plots depict the dose-response relationship between the logarithm of peptide concentration and GIPR activation. The corresponding EC₅₀ values are shown in the table below the plots. FIG. 9 compares the different number of carbons from the amide bond to the quaternary carbon (T1, T2, T3, and T4).

FIG. 10 shows activation of GIPR by GLP-1 analogues with different amide positions of the side chain modification at backbone position 16, revealed by cAMP production. The scatter plots depict the dose-response relationship between the logarithm of peptide concentration and GIPR activation. The corresponding EC₅₀ values are shown in the table below the plots. FIG. 10 compares different amide positions with B4T2, B3T3, and B2T4 side chains.

FIGS. 11-13 show activation of GIPR by GLP-1 analogues with various appendages of the side chain modification at the same length (GLP-1* SEQ ID No: 6, B4T2 OMe, 4-(4-hydroxy)phenyl butyric acid, 4-phenyl butyric acid, and 4-(4-chloro)phenyl butyric acid, 4-(3-indole) butyric acid, 4-(2-pyrene) butyric acid, and 4-cyclohexane butyric acid)) at backbone position 16, revealed by the CAMP production. The scatter plot depicts the dose-response relationship between the logarithm of peptide concentration and GIPR activation. The corresponding EC₅₀ values are shown in the table below the plot.

FIG. 14 shows activation of GIPR by GLP-1 analogues with varying groups ortho to the hydroxyl group on the bisphenol at backbone position 16, revealed by cAMP production. The scatter plots depict the dose-response relationship between the logarithm of peptide concentration and GIPR activation. The corresponding EC₅₀ values are shown in the table below the plots.

FIG. 15 shows activation of GIPR by GLP-1 analogues with different chirality at the sidechain at backbone position 16, revealed by cAMP production. The scatter plots depict the dose-response relationship between the logarithm of peptide concentration and GIPR activation. The corresponding EC₅₀ values are shown in the table below the plots.

FIG. 16 shows activation of GLP-1R by GIP analogues with side chain modification at various backbone positions (9, 10, 11, 12, 13). FIG. 17 presents the results from the CAMP production assay and FIG. 18 presents the results from the β-arrestin-2 recruitment assay for GIP* (SEQ ID No: 7) at the GIPR. The scatter plots depict the dose-response relationship between the logarithm of peptide concentration and GIPR activation. The corresponding EC₅₀ values are shown in the table below the plots.

FIGS. 19-20 show activation of GLP-1R by GIP analogues with varying length of the side chain modification at backbone position 10, revealed by cAMP production. The scatter plots depict the dose-response relationship between the logarithm of peptide concentration and GLP-1R activation. The corresponding EC₅₀ values are shown in the table below the plots. FIG. 19 compares different number of carbons from the amide bond to the quaternary carbon (T1, T2, T3, and T4). FIG. 20 compares the different number of carbons from the backbone to the amide bond (B1, B2, B3, and B4).

FIG. 21 compares GIP analogues with various appendages of the side chain modification at backbone position 10 (3-(4-hydroxy)phenyl propanoic acid, 3-(3,4 dihydroxy)phenyl propanoic acid, 3-(3-indole) propanoic acid, 3-(4-fluoro)phenyl propanoic acid, and diphenyl acetic acid).

FIG. 22 shows activation of GLP-1R by GIP analogues with different chirality at the backbone. The scatter plots depict the dose-response relationship between the logarithm of peptide concentration and GLP-1R activation. The corresponding EC₅₀ values are shown in the table below the plots.

### DETAILED DESCRIPTION

### Abbreviations and Definitions

Aib=2-aminoisobutyric acid (i.e., 2-methylalanine)
α-N-Fmoc=fluorenylmethoxycarbonyl-N—R.
Alloc=allyloxycarbonyl:

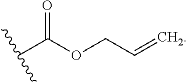 [SMILES: *C(=O)OCC=C]

CHCA=α-cyano-4-hydroxycinnamic acid.
DCE=dichloroethane.
DIC=N,N′-diisopropylcarbodiimide.
DMF=N,N-dimethylformamide.
DPBS=Dulbecco's phosphate buffered saline.
EtOAc=ethyl acetate.
Fatty acid-like side chain=a side chain selected from the group consisting of:

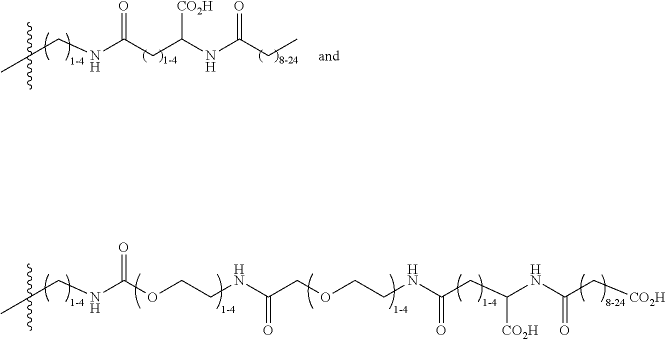

FBS=fetal bovine serum.
GIP=glucose-dependent insulinotropic polypeptide, a pro-peptide thereof, a fragment thereof at least about 15 residues long, at least about 20 residues long, or at least about 25 residues long, and analogs of the foregoing having at least 80%, at least 85%, at least 90%, at least 95%, or at least about 99% sequence identity thereto, C-terminal amides thereof, and salts of the foregoing.
GLP-1=glucagon-like peptide-1, a pro-peptide thereof, a fragment thereof at least about 15 residues long, at least about 20 residues long, or at least about 25 residues long, and analogs of the foregoing having at least 80%, at least 85%, at least 90%, at least 95%, or at least about 99% sequence identity thereto, C-terminal amides thereof, and salts of the foregoing.

Heterocyclyl groups include aromatic (also referred to as heteroaryl, see below) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

The terms “identity” and “identical” and their variants, as used herein, when used in reference to two or more amino acid sequences (or nucleotide sequences), refer to similarity in sequence of the two or more sequences (i.e., polypeptide or nucleotide sequences). In the context of two or more homologous sequences, the percent identity or homology of the sequences or sub-sequences thereof indicates the percentage of all monomeric units (amino acid residues or nucleotide bases) that are the same (e.g., about 70% identity, preferably 75%, 80%, 85%, 90%, 95%, 98% or 99% identity). The percent identity can be over a specified region, when compared and aligned for maximum correspondence over a comparison window, or over a designated region as measured using a BLAST or BLAST 2.0 sequence comparison algorithms with pre-defined default parameters, or by manual alignment and visual inspection. Sequences are said to be “substantially identical” when there is at least 85% identity at the amino acid level or at the nucleotide level. Conventional and well-known algorithm for determining percent sequence identity and sequence similarity are the BLAST and BLAST 2.0 algorithms, which are described in Altschul et al, Nuc. Acids Res. 25:3389-3402 (1997). Other methods include the algorithms of Smith & Waterman, Adv. Appl. Math. 2:482 (1981), and Needleman & Wunsch, J. Mol. Biol. 48:443 (1970), etc. Various versions of the BLAST program are made available online to the public free of charge from the National Library of Medicine of the National Center for Biotechnology Information at blast.ncbi.nlm.nih.gov/Blast.cgi.

Oxyma=ethyl (hydroxyimino) cyanoacetate.

“Pharmaceutically suitable salt”=any acid or base addition salt whose counter-ions are non-toxic to the patient in pharmaceutical doses of the salts, so that the beneficial inhibitory effects inherent in the free base or free acid are not vitiated by side effects ascribable to the counter-ions. A host of pharmaceutically suitable salts are well known in the art. For basic active ingredients, all acid addition salts are useful as sources of the free base form even if the particular salt, per se, is desired only as an intermediate product as, for example, when the salt is formed only for purposes of purification, and identification, or when it is used as intermediate in preparing a pharmaceutically suitable salt by ion exchange procedures. Pharmaceutically suitable salts include, without limitation, those derived from mineral acids and organic acids, explicitly including hydrohalides, e.g., hydrochlorides and hydrobromides, sulphates, phosphates, nitrates, sulphamates, acetates, citrates, lactates, tartrates, malonates, oxalates, salicylates, propionates, succinates, fumarates, maleates, methylene bis b hydroxynaphthoates, gentisates, isethionates, di-p-toluoyltartrates, methane sulphonates, ethanesulphonates, benzenesulphonates, p-toluenesulphonates, cyclohexylsulphamates, quinates, and the like. Base addition salts include those derived from alkali or alkaline earth metal bases or conventional organic bases, such as triethylamine, pyridine, piperidine, morpholine, N-methylmorpholine, and the like. See, for example, “Handbook of Pharmaceutical Salts-Properties, Selection, and Use,” 2^{nd} Revised Ed., P. H. Stahl and C. G. Wermuch, Eds., © 2011, Wiley-VCH (Zurich, Switzerland), ISBN: 978-3906390512.

Polycyclic aromatic hydrocarbon=naphthalene, biphenyl, fluorene, anthracene, phenanthrene, phenalene, tetracene, chrysene, triphenylene, pyrene, and the like.

Reference to a certain element, such as hydrogen, includes all isotopes of that element. For example, if an R group is defined to include hydrogen, it also includes deuterium and tritium. Compounds comprising radioisotopes such as tritium, C¹⁴, p³² and S³⁵ are thus within the scope of the present disclosure. Procedures for inserting such labels into the compounds disclosed herein will be readily apparent to those skilled in the art based on the disclosure herein.

In general, “substituted” refers to an organic group (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., Cl. F, Br, and I); hydroxyls; alkoxy, alkenoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups.

TFA=trifluoroacetic acid.

TIPS=triisopropylsilane.

### Dural GLP-1 Receptor and GIP Receptor Agonists:

Disclosed herein is a method of making dual GLP-1 receptor and GIP receptor agonists. The method comprising modifying at least one side chain of a residue in GLP-1 and/or GIP, to yield a modified GLP-1 and/or GIP, wherein the modified GLP-1 and GIP are agonists of both GLP-1 receptor and GIP receptor. Also disclosed herein are the resulting compounds, along with pharmaceutical compositions containing the compounds for use (primarily) to treat Type 2 diabetes and also (secondarily) to treat obesity.

Class B G protein-coupled receptors (GPCRs) are activated by long polypeptide hormones. Several of these receptors are targets of drugs, which function by activating a specific class B GPCR. Peptides that activate the glucagon receptor (GCGR) are used to treat hypoglycemic shock in type I diabetes patients. Peptides that activate the glucagon-like peptide-1 receptor (GLP-1R) are used to treat type 2 diabetes (T2D) and obesity. Peptides that activate the GLP-2R are used to treat short-bowel syndrome. Peptides that activate the gastic inhibitory peptide receptor (GIPR) have also been explored for clinical use to treat T2D and obesity.

It has been suggested that T2D/obesity treatment might be improved with agonists that activate more than one among three related receptors: the GLP-1R, GIPR and GCGR. Dual GLP-1R/GIPR and GLP-1R/GCGR agonists and triple GLP-1R/GIPR/GCGR agonists have been explored.

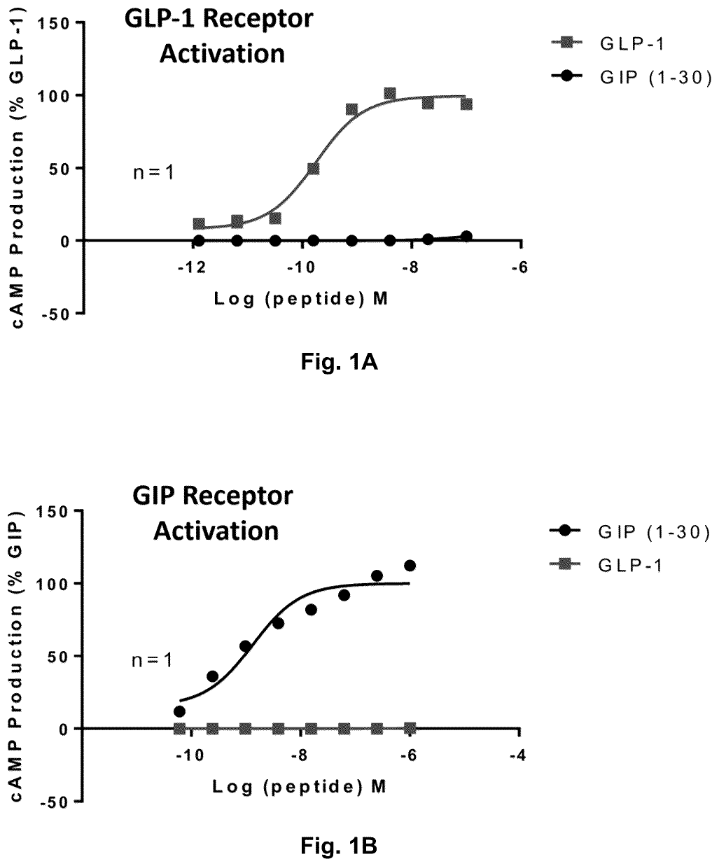

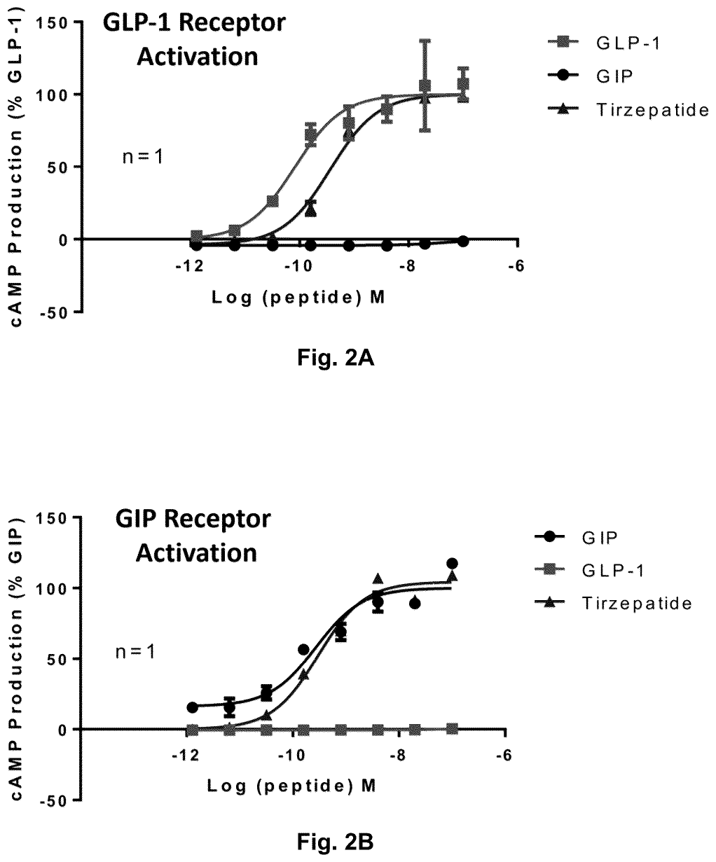

GLP-1 and GIP each contain 29 or more residues, but their sequences differ. GLP-1 does not activate the GIPR, and GIP does not activate the GLP-1R (FIGS. 1A-1B). The drug tirzepatide (TZP; SEQ ID NO: 5) was developed by combining residues from GLP-1 and GIP and have been shown to be a dual GLP-1R/GIPR (FIGS. 2A-2B). The sequence of TZP has many differences relative to the sequence of GLP-1 or GIP.

In the present disclosure, we have discovered that a single change in the sequence of GLP-1 can “turn on” agonist activity at the GIPR. In addition, we have discovered that a single change in the sequence of GIP can “turn on” agonist activity at the GLP-1R. In each case, the single change involves replacing a native amino acid residue in the middle region of the hormone with an unnatural residue that has a large moiety at its end. In one version of the disclosure, the large moiety contains two phenol units. Without being limited to any underlying biological mechanism or phenomenon, the moiety on the GLP-1 derivative may engage specific sites on the surface of the GIPR, and that the large moiety on the GIP derivative may engage specific sites on the surface of the GLP-1R. In each case, these non-natural contacts promote receptor activation.

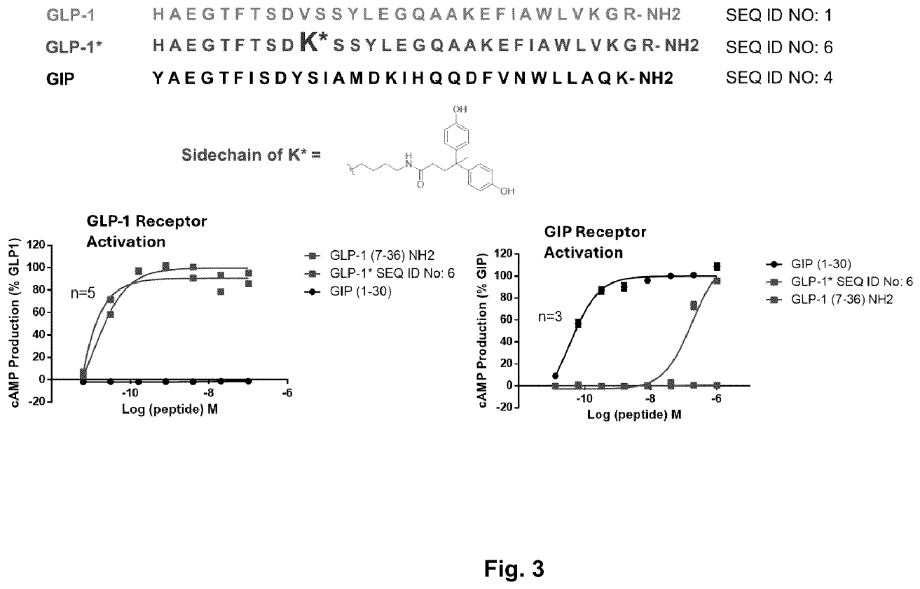

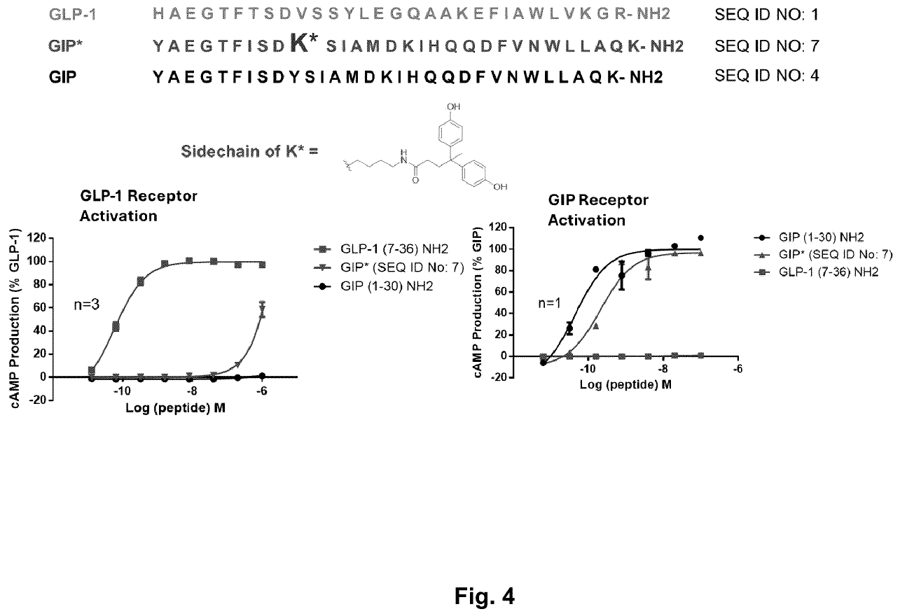

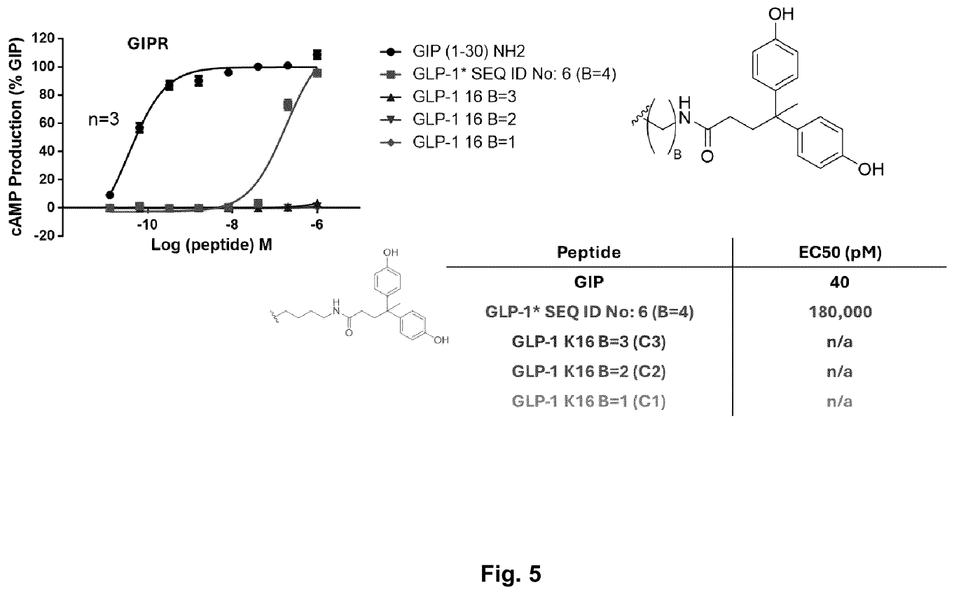

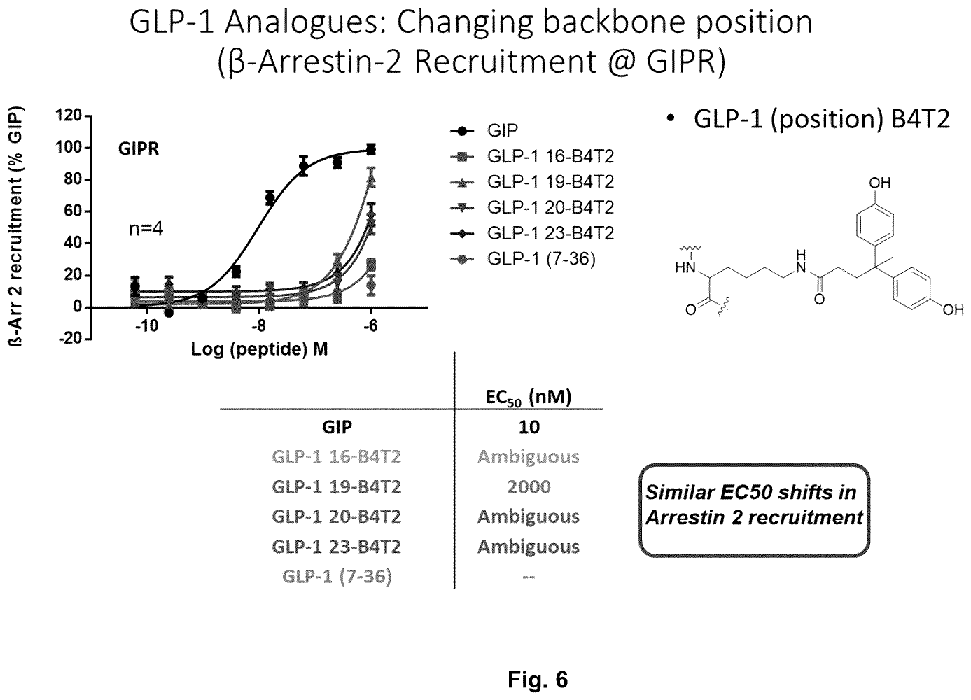

FIGS. 3 and 4 show representative examples of the new dual agonists, one GLP-1 derivative with an activating appendage (GLP-1*; SEQ ID NO: 6; FIG. 3), and one GIP derivative with an activating appendage (GIP*; SEQ ID NO: 7; FIG. 4). As shown in the figures, GLP-1* and GIP* are agonists of both GLP-1 receptor and GIP receptor. It has also been shown that the GIP receptor agonist activity of GLP-1* can be altered by adjusting the length of the side chain modification with the C₄-length side chain having greater GIP receptor agonist activity (FIG. 5). Additional exemplary dual agonists were synthesized with appendage placement at various positions in each hormone and with a variety of large moieties.

The “activating appendage” strategy may be a general method for developing new peptides with polypharmacology (i.e., the ability to activate more than one receptor). Because the sequences of the dual agonists are closer to those of the native peptide hormones relative to TZP, the compounds might be less prone than TZP to eliciting neutralizing antibodies in patients. In addition, the method may allow one to generate new compounds with activity profiles that are not readily accessible in other ways.

### Pharmaceutical Compositions:

Also disclosed herein are pharmaceutical compositions comprising one or more of the GIP and/or GLP-1 analogs or a pharmaceutically suitable salt thereof as described herein. More specifically, the pharmaceutical composition may comprise one or more of the GIP and/or GLP-1 analogs as well as a standard, well-known, non-toxic pharmaceutically suitable carrier, adjuvant or vehicle such as, for example, phosphate buffered saline, water, ethanol, polyols, vegetable oils, a wetting agent or an emulsion such as a water/oil emulsion. The composition may be in either a liquid, solid or semi-solid form. For example, the composition may be in the form of a tablet, capsule, ingestible liquid or powder, injectable, suppository, or topical ointment or cream. Proper fluidity can be maintained, for example, by maintaining appropriate particle size in the case of dispersions and by the use of surfactants. It may also be desirable to include isotonic agents, for example, sugars, sodium chloride, and the like. Besides such inert diluents, the composition may also include adjuvants, such as wetting agents, emulsifying and suspending agents, sweetening agents, flavoring agents, perfuming agents, and the like.

Suspensions, in addition to the active compounds, may comprise suspending agents such as, for example, ethoxylated isostearyl alcohols, polyoxyethylene sorbitol and sorbitan esters, microcrystalline cellulose, aluminum metahydroxide, bentonite, agar-agar and tragacanth or mixtures of these substances.

Solid dosage forms such as tablets and capsules can be prepared using techniques well known in the art of pharmacy. For example, GIP and GLP-1 analogs produced as described herein can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant GLP-1 analog.

For intravenous, intramuscular, and subcutaneous administration, the GIP and GLP-1 analogs may be incorporated into commercial formulations such as Intralipid©-brand fat emulsions for injection. (“Intralipid” is a registered trademark of Fresenius Kabi AB, Uppsalla, Sweden.) Where desired, the individual components of the formulations may be provided individually, in kit form, for single or multiple doses. A typical injectable dose of a representative GIP or GLP-1 analog as described herein is from about 0.01 mg to about 100 mg daily and is preferably from 0.2 mg to 3.0 mg daily. Dosages above and below these stated ranges are specifically within the scope of the claims.

Possible routes of administration of the pharmaceutical compositions include, for example, enteral (e.g., oral and rectal) and parenteral. For example, a liquid preparation may be administered, for example, orally or rectally. Additionally, a homogenous mixture can be completely dispersed in water, admixed under sterile conditions with physiologically acceptable diluents, preservatives, buffers or propellants in order to form a spray or inhalant. The route of administration will, of course, depend upon the desired effect and the medical state of the subject being treated. The dosage of the composition to be administered to the patient may be determined by one of ordinary skill in the art and depends upon various factors such as weight of the patient, age of the patient, immune status of the patient, etc., and is ultimately at the discretion of the medical professional administering the treatment.

With respect to form, the composition may be, for example, a solution, a dispersion, a suspension, an emulsion, or a sterile powder which is then reconstituted. The composition may be administered in a single daily or weekly dose or multiple daily or weekly doses.

The present disclosure also includes treating hyperglycemic disorders and obesity in mammals, including humans, by administering an anti-hyperglycemic-effective amount of one or more the GIP and/or GLP-1 analogs described herein. In particular, the compositions of the present invention may be used to treat diabetic conditions of any and all description. Additionally, the compositions of the present invention may also be used to prevent the apoptotic death of β cells in the pancreas. To the extent the compositions impart a feeling of satiation, the compositions may also be used to treat obesity and to ease weight loss.

The above-described pharmaceutical compositions may be utilized in connection with non-human animals, both domestic and non-domestic, as well as humans.

### Exemplary Compounds:

A systematic shorthand has been developed to name the subject compounds quickly and unambiguously. The parent peptide is named first (e.g., GLP-1, GIP, etc.), followed by the numerical position of the residue modified. The subscript/notation “B” represents the number of carbon atoms between the backbone alpha-carbon of the modified residue and the amide bond in the modified side chain. The subscript/notation “T” represents the number of carbon atoms from the amide bond to the quaternary carbon atom (or other carbon atom, as noted) of the modified side chain:

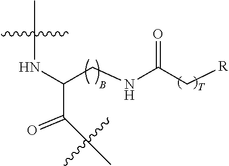

Thus, for example, the compound GLP-1 16-B4 T2 is the GLP-1 polypeptide (SEQ ID NO: 1) having the following modified side chain at position 16:

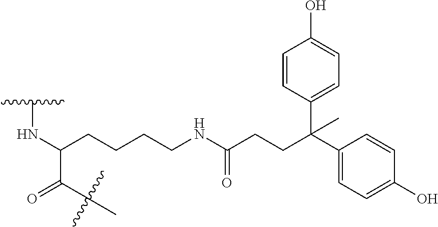 [SMILES: *NC(CCCCNC(=O)CCC(C)(c1ccc(O)cc1)c1ccc(O)cc1)C(*)=O]

(this derivative of GLP-1 is GLP-1* (SEQ ID NO: 6). The shorthand may include a further suffix to define the nature of the terminal moiety on the modified side chain. Names without a suffix have a bisphenol A terminal group. The hydroxy groups on the bisphenol may be substituted with other groups at other positions (as defined herein). The bisphenol A terminal moiety (shown immediately above) may be replaced with other types of bisphenols or fused aromatic groups as defined herein, e.g., bisphenol AP, bisphenol B, bisphenol C, bisphenol G, etc.).

Using the shorthand notation described above, exemplary modified versions of GLP-1 (SEQ ID NO: 1) that have been made according to the present disclosure are listed in Table 1.

TABLE 1
Exemplary Working GLP-1 Compounds Made and Evaluated:

| Compound Shorthand Name | Modified Side Group |
|---|---|
| GLP-1 12 B4T2 | 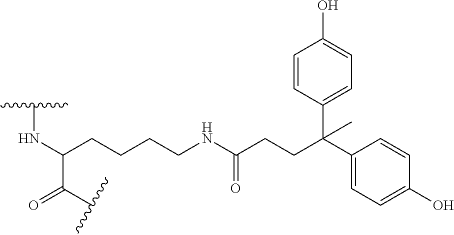 [SMILES: *NC(CCCCNC(=O)CCC(C)(c1ccc(O)cc1)c1ccc(O)cc1)C(*)=O] |
| GLP-1 16 B1 T2 | 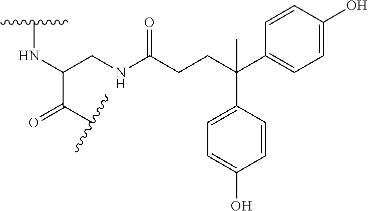 [SMILES: *NC(CNC(=O)CCC(C)(c1ccc(O)cc1)c1ccc(O)cc1)C(*)=O] |
| GLP-1 16 B2 T2 | 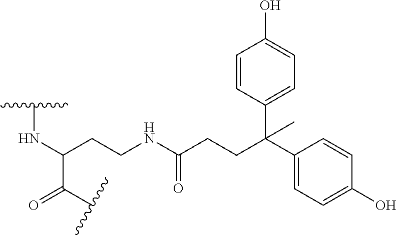 [SMILES: *NC(CCNC(=O)CCC(C)(c1ccc(O)cc1)c1ccc(O)cc1)C(*)=O] |
| GLP-1 16 B2 T4 | 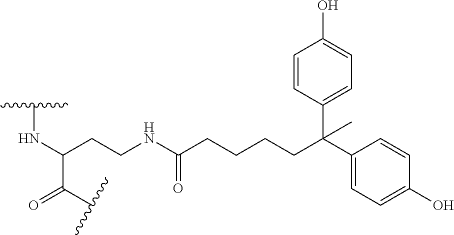 [SMILES: *NC(CCNC(=O)CCCCC(C)(c1ccc(O)cc1)c1ccc(O)cc1)C(*)=O] |
| GLP-1 16 B3 T2 | 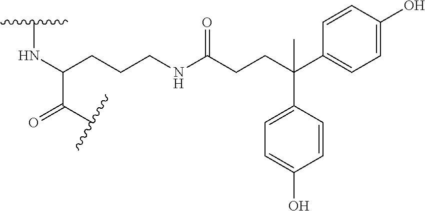 [SMILES: *NC(CCCNC(=O)CCC(C)(c1ccc(O)cc1)c1ccc(O)cc1)C(*)=O] |
| GLP-1 16 B3 T3 | 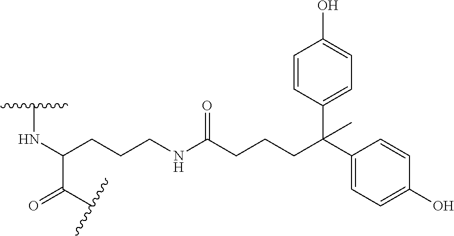 [SMILES: *NC(CCCNC(=O)CCCC(C)(c1ccc(O)cc1)c1ccc(O)cc1)C(*)=O] |
| GLP-1 16 B4 T1 | 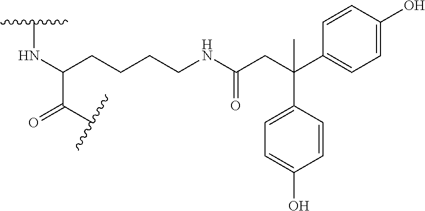 [SMILES: *NC(CCCCNC(=O)CC(C)(c1ccc(O)cc1)c1ccc(O)cc1)C(*)=O] |
| GLP-1 16 B4 T2 | 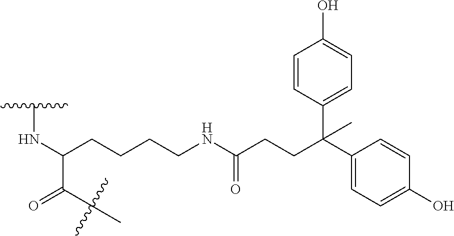 [SMILES: *NC(CCCCNC(=O)CCC(C)(c1ccc(O)cc1)c1ccc(O)cc1)C(*)=O] |
| GLP-1 16 B4 T3 | 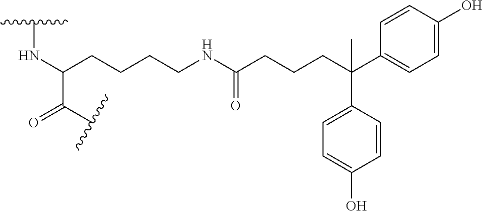 [SMILES: *NC(CCCCNC(=O)CCCC(C)(c1ccc(O)cc1)c1ccc(O)cc1)C(*)=O] |
| GLP-1 16 B4 T4 | 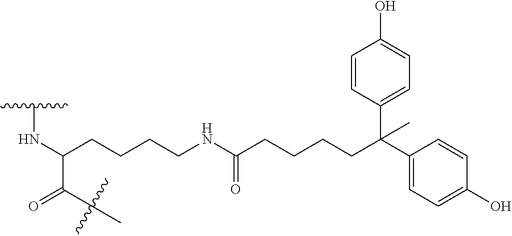 [SMILES: *NC(CCCCNC(=O)CCCCC(C)(c1ccc(O)cc1)c1ccc(O)cc1)C(*)=O] |
| GLP-1 16 4-(4-hydroxyphenyl) butyric acid | 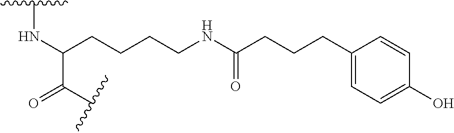 [SMILES: *NC(CCCCNC(=O)CCCc1ccc(O)cc1)C(*)=O] |
| GLP-1 16 B4 T2-OMe | 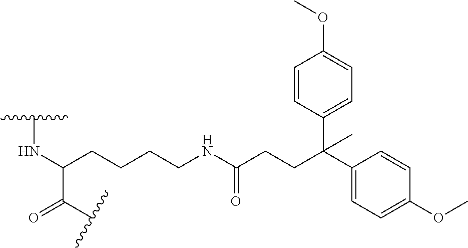 [SMILES: *NC(CCCCNC(=O)CCC(C)(c1ccc(OC)cc1)c1ccc(OC)cc1)C(*)=O] |
| GLP-1 16 4-phenyl butyric acid | 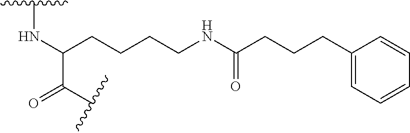 [SMILES: *NC(CCCCNC(=O)CCCc1ccccc1)C(*)=O] |
| GLP-1 16 4-(4-chlorophenyl) butyric acid | 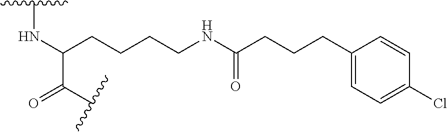 [SMILES: *NC(CCCCNC(=O)CCCc1ccc(Cl)cc1)C(*)=O] |
| GLP-1 16 4-(3-indole) butyric acid | 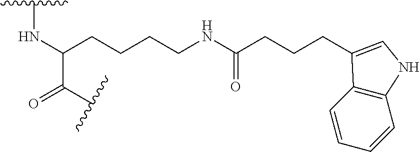 [SMILES: *NC(CCCCNC(=O)CCCc1c[nH]c2ccccc12)C(*)=O] |
| GLP-1 16 4-(2-pyrene) butyric acid | 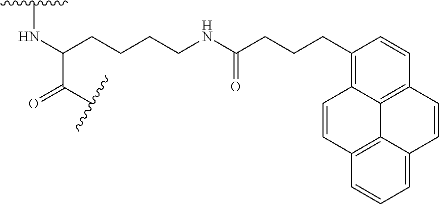 [SMILES: *NC(CCCCNC(=O)CCCc1ccc2ccc3cccc4ccc1c2c34)C(*)=O] |
| GLP-1 16 4-cyclohexane butyric acid | 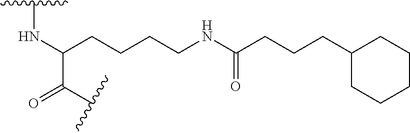 [SMILES: *NC(CCCCNC(=O)CCCC1CCCCC1)C(*)=O] |
| GLP-1 16 B4T2 X = OH | 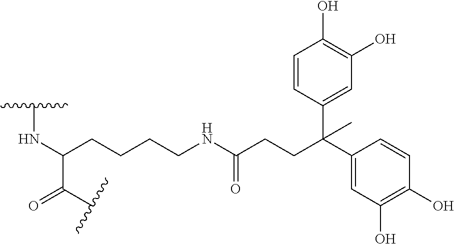 [SMILES: *NC(CCCCNC(=O)CCC(C)(c1ccc(O)c(O)c1)c1ccc(O)c(O)c1)C(*)=O] |
| GLP-1 16 B4T2 X = F | 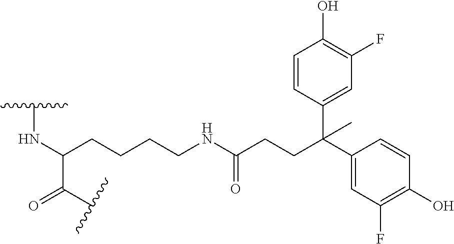 [SMILES: *NC(CCCCNC(=O)CCC(C)(c1ccc(O)c(F)c1)c1ccc(O)c(F)c1)C(*)=O] |
| GLP-1 16 B4 T2 X = Cl | 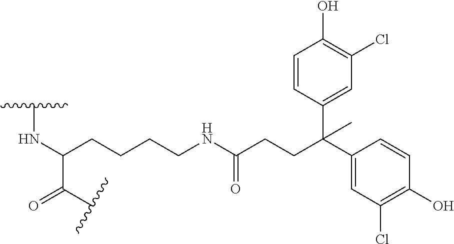 [SMILES: *NC(CCCCNC(=O)CCC(C)(c1ccc(O)c(Cl)c1)c1ccc(O)c(Cl)c1)C(*)=O] |
| GLP-1 18 B4T2 | 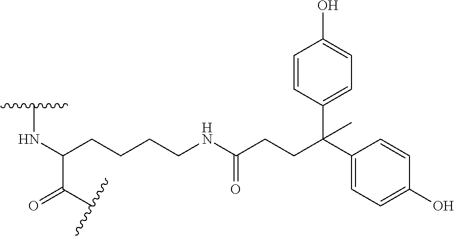 [SMILES: *NC(CCCCNC(=O)CCC(C)(c1ccc(O)cc1)c1ccc(O)cc1)C(*)=O] |
| GLP-1 22 B4T2 | 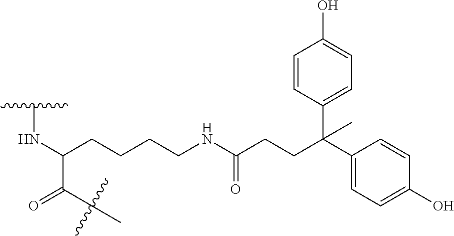 [SMILES: *NC(CCCCNC(=O)CCC(C)(c1ccc(O)cc1)c1ccc(O)cc1)C(*)=O] |
| GLP-1 26 B4T2 | 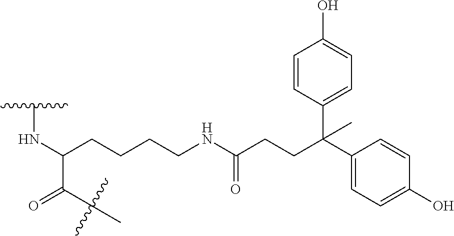 [SMILES: *NC(CCCCNC(=O)CCC(C)(c1ccc(O)cc1)c1ccc(O)cc1)C(*)=O] |

Using the shorthand notation described above, exemplary modified versions of GIP (SEQ ID NO: 4) that have been made according to the present disclosure are listed in Table 2.

TABLE 2
Exemplary Working GIP Compounds Made and Evaluated:

| Compound Shorthand Name | Modified Side Group |
|---|---|
| GIP 9 B4 T2 | 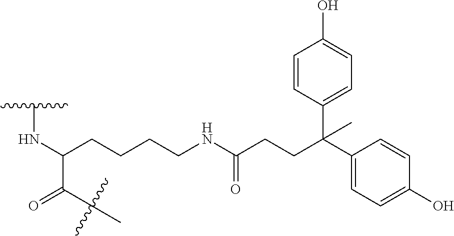 [SMILES: *NC(CCCCNC(=O)CCC(C)(c1ccc(O)cc1)c1ccc(O)cc1)C(*)=O] |
| GIP 10 B4 T2 | 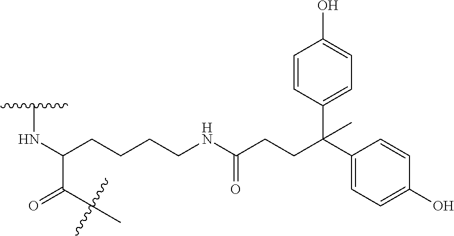 [SMILES: *NC(CCCCNC(=O)CCC(C)(c1ccc(O)cc1)c1ccc(O)cc1)C(*)=O] |
| GIP 10 B1 T2 | 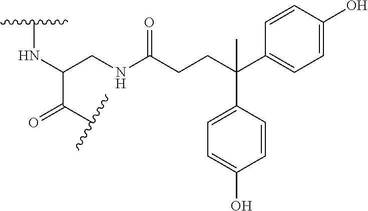 [SMILES: *NC(CNC(=O)CCC(C)(c1ccc(O)cc1)c1ccc(O)cc1)C(*)=O] |
| GIP 10 B2 T2 | 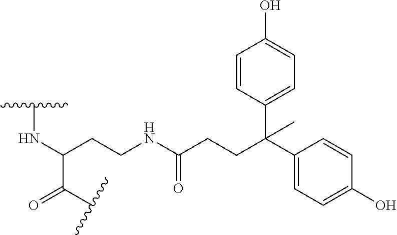 [SMILES: *NC(CCNC(=O)CCC(C)(c1ccc(O)cc1)c1ccc(O)cc1)C(*)=O] |
| GIP 10 B2 T4 | 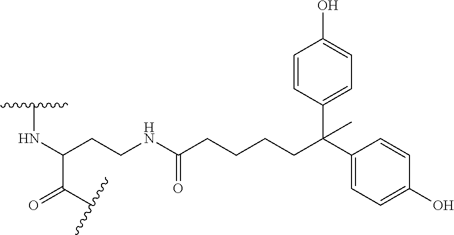 [SMILES: *NC(CCNC(=O)CCCCC(C)(c1ccc(O)cc1)c1ccc(O)cc1)C(*)=O] |
| GIP 10 B3 T2 | 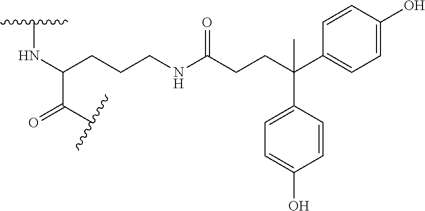 [SMILES: *NC(CCCNC(=O)CCC(C)(c1ccc(O)cc1)c1ccc(O)cc1)C(*)=O] |
| GIP 10 B3 T3 | 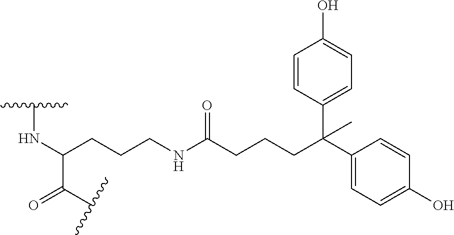 [SMILES: *NC(CCCNC(=O)CCCC(C)(c1ccc(O)cc1)c1ccc(O)cc1)C(*)=O] |
| GIP 10 B4 T1 | 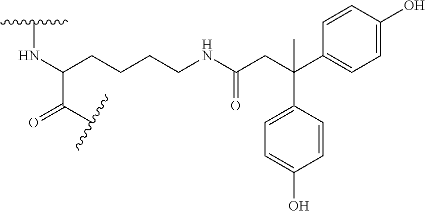 [SMILES: *NC(CCCCNC(=O)CC(C)(c1ccc(O)cc1)c1ccc(O)cc1)C(*)=O] |
| GIP 10 B4 T2 | 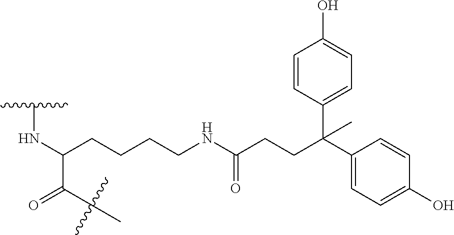 [SMILES: *NC(CCCCNC(=O)CCC(C)(c1ccc(O)cc1)c1ccc(O)cc1)C(*)=O] |
| GIP 10 B4 T3 | 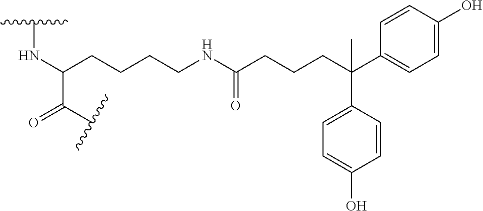 [SMILES: *NC(CCCCNC(=O)CCCC(C)(c1ccc(O)cc1)c1ccc(O)cc1)C(*)=O] |
| GIP 10 B4 T4 | 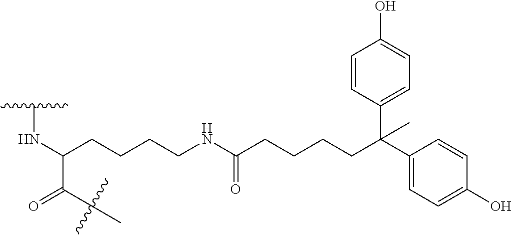 [SMILES: *NC(CCCCNC(=O)CCCCC(C)(c1ccc(O)cc1)c1ccc(O)cc1)C(*)=O] |
| GIP 10 3-(4-hydroxy)phenyl propanoic acid | 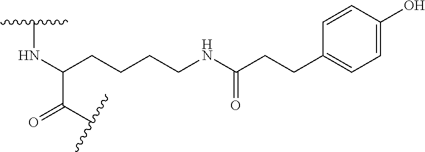 [SMILES: *NC(CCCCNC(=O)CCc1ccc(O)cc1)C(*)=O] |
| GIP 10 3-(3,4-dihydroxy)phenyl propanoic acid | 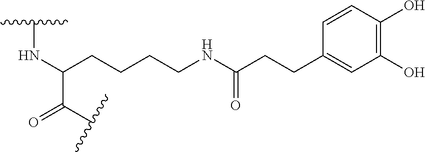 [SMILES: *NC(CCCCNC(=O)CCc1ccc(O)c(O)c1)C(*)=O] |
| GIP 10 3-(3-indole) propanoic acid | 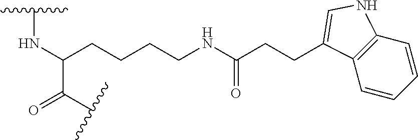 [SMILES: *NC(CCCCNC(=O)CCc1c[nH]c2ccccc12)C(*)=O] |
| GIP 10 3-(4-fluoro)phenyl propanoic acid | 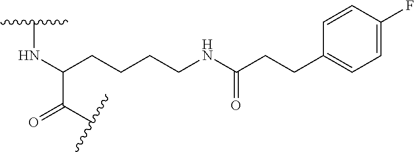 [SMILES: *NC(CCCCNC(=O)CCc1ccc(F)cc1)C(*)=O] |
| GIP 10 diphenyl acetic acid | 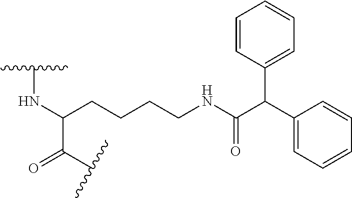 [SMILES: *NC(CCCCNC(=O)C(c1ccccc1)c1ccccc1)C(*)=O] |
| GIP 11 B4 T2 | 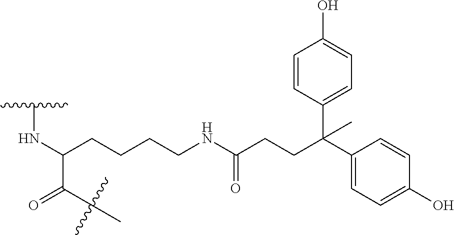 [SMILES: *NC(CCCCNC(=O)CCC(C)(c1ccc(O)cc1)c1ccc(O)cc1)C(*)=O] |
| GIP 12 B4 T2 | 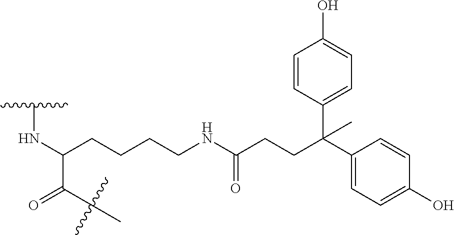 [SMILES: *NC(CCCCNC(=O)CCC(C)(c1ccc(O)cc1)c1ccc(O)cc1)C(*)=O] |
| GIP 13 B4 T2 | 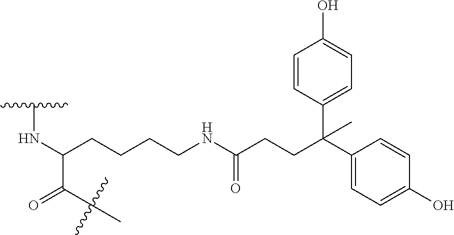 [SMILES: *NC(CCCCNC(=O)CCC(C)(c1ccc(O)cc1)c1ccc(O)cc1)C(*)=O] |

### Evaluation of the Exemplary Compounds:

β-arrestin recruitment and cAMP production assays were used to evaluate activation of the GIP receptor and GLP-1 receptor by the GLP-1 and GIP analogues. Upon activation of the GIPR and GLP-1R, β-arrestin is recruited to the receptor, and intracellular cAMP levels increase.

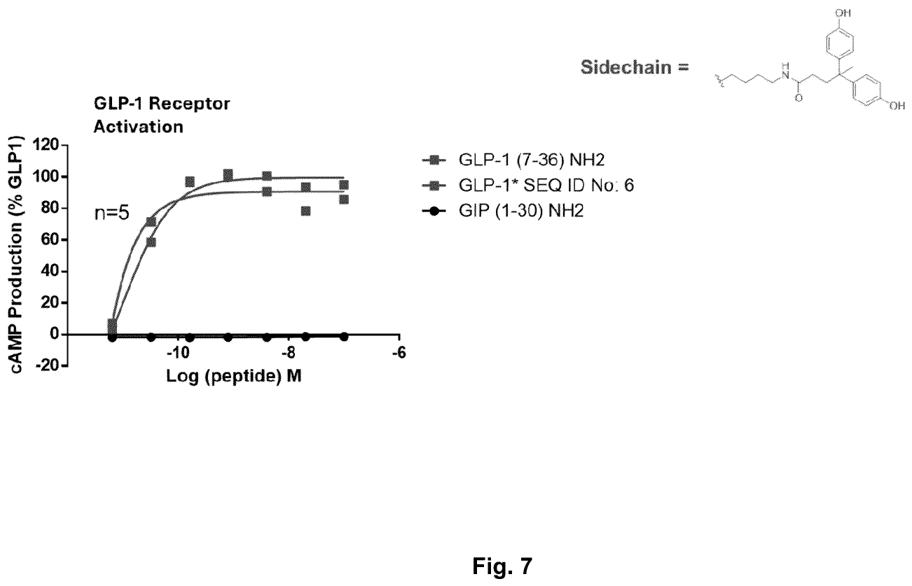

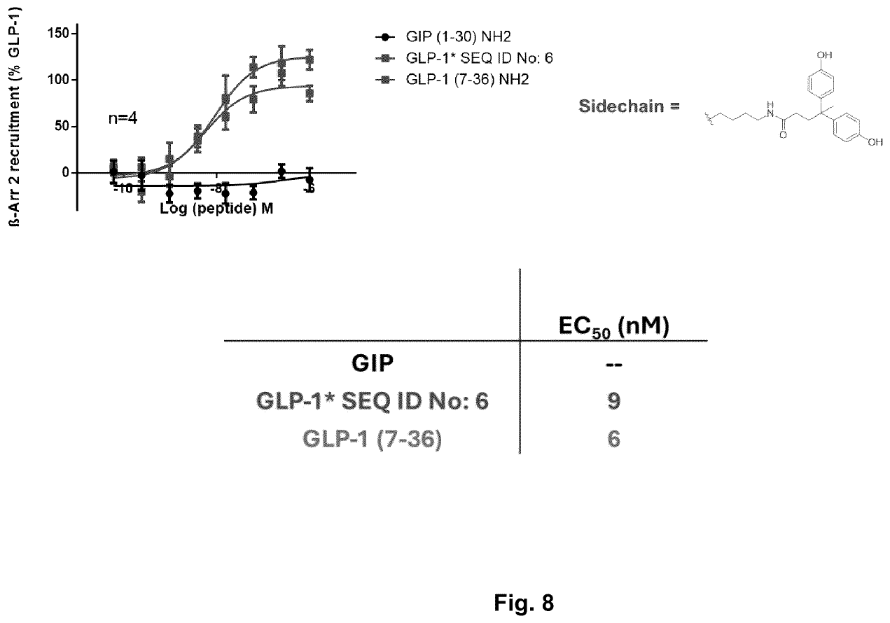

FIG. 6 compares the potency of GLP-1 analogues with side chain modification at various backbone positions for activating the GIPR. The best potency was observed for the activation of GIPR by the GLP-1 analogue with modification at backbone position 16 (SEQ ID NO: 6). No significant potency changes, in terms of cAMP production or β-arrestin recruitment, were observed for the activation of GLP-1R by the GLP-1 analogue with modification at backbone position 16 (SEQ ID NO: 6) (FIGS. 7-8).

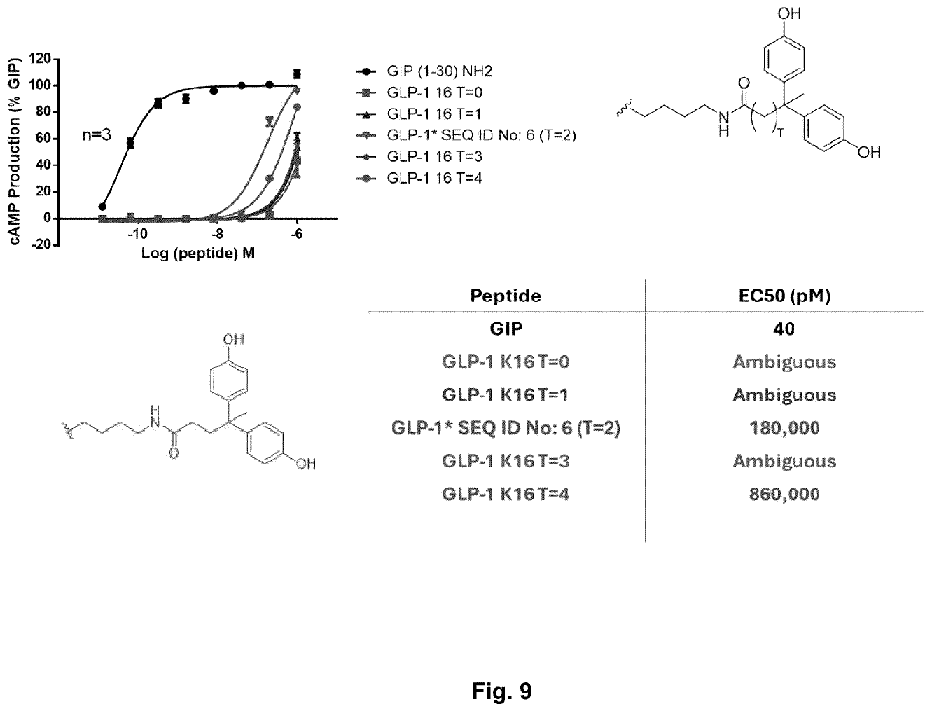

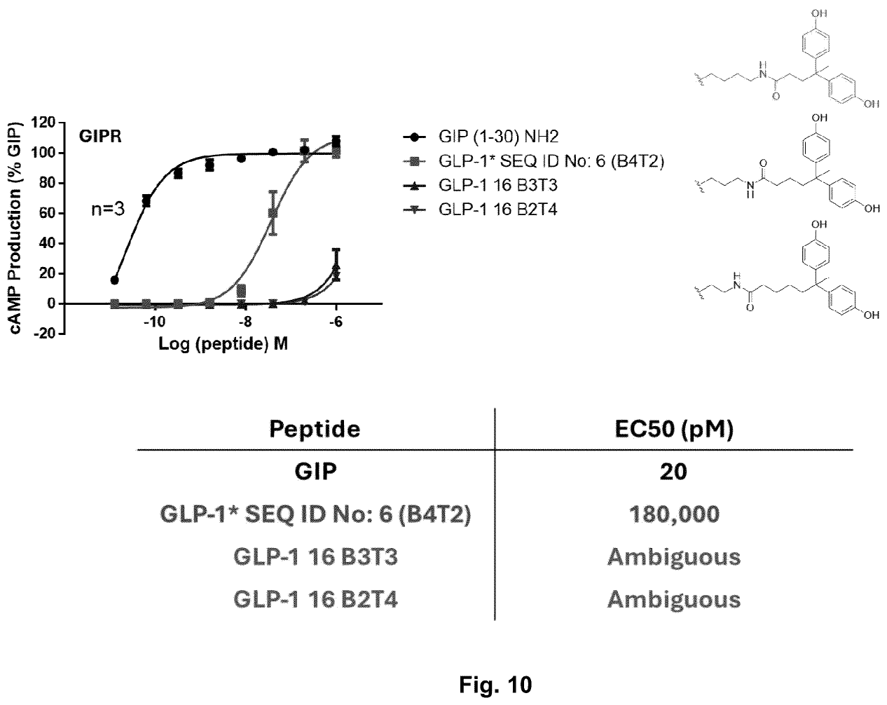

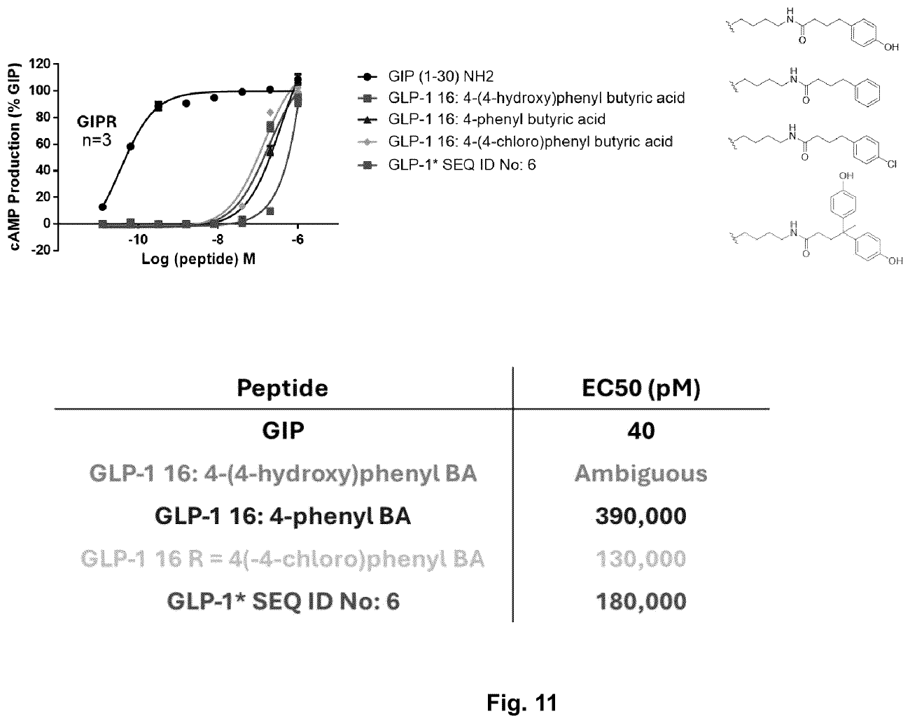

The effects of the side chain length of the modification at backbone position 16 of GLP-1 were further examined. Potency changes were observed for the activation of GIPR by GLP-1 analogues with varying number of carbons from the amide bond to the quaternary carbon (the number of “T”; FIG. 9), while the number of carbons from the backbone to the amide bond (the number of “B”) significantly influenced the potency of the GLP-1 analogues, with B4 showing greater activity than B1, B2, and B3 (FIG. 5).). The different numbers of “B” result in different amide positions relative to the peptide backbone. As shown in FIG. 10, the amide position is important for the activity of GLP-1 analogues at the GIPR, given the same side chain length. The amide group positioned after B4 exhibited greater activity than those positioned after B2 and B3.

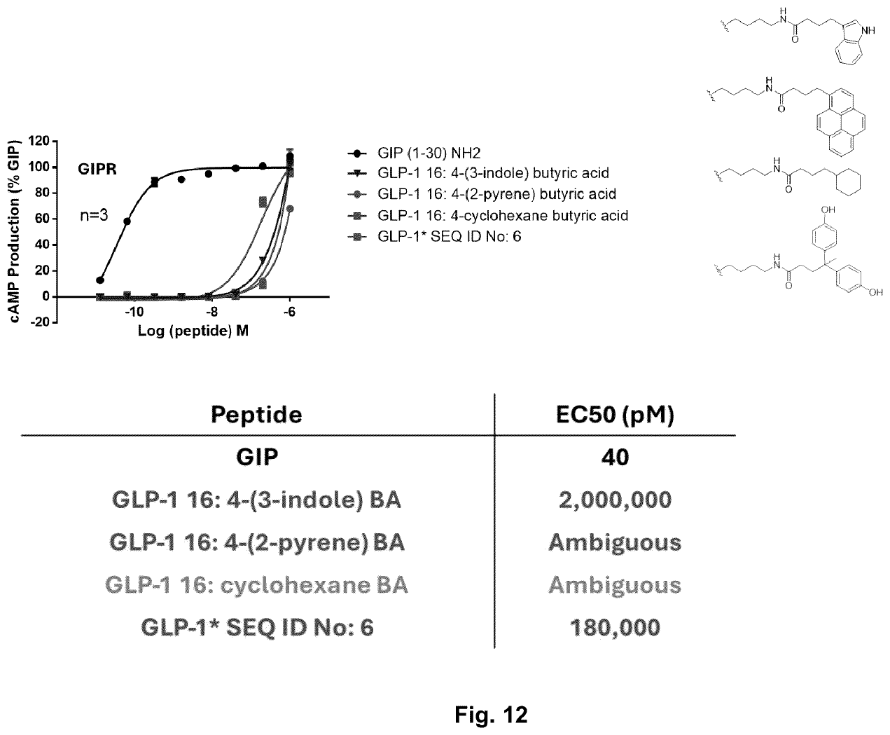

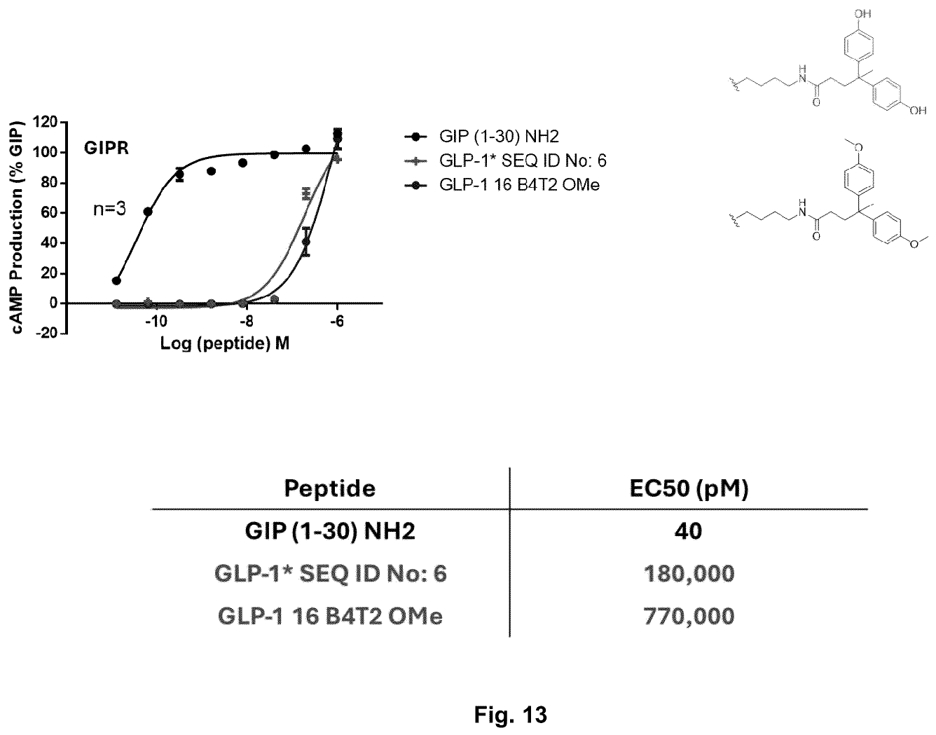

FIGS. 11-13 show the results of side chain modification with various appendages at backbone position 16 of GLP-1 on activity at the GIPR. As shown in the figures, modification with appendages-GLP-1* SEQ ID No: 6 (B4T2), 4-(4-chloro)phenyl butyric acid, and B4T2 OMe exhibited greater activity than other tested appendages.

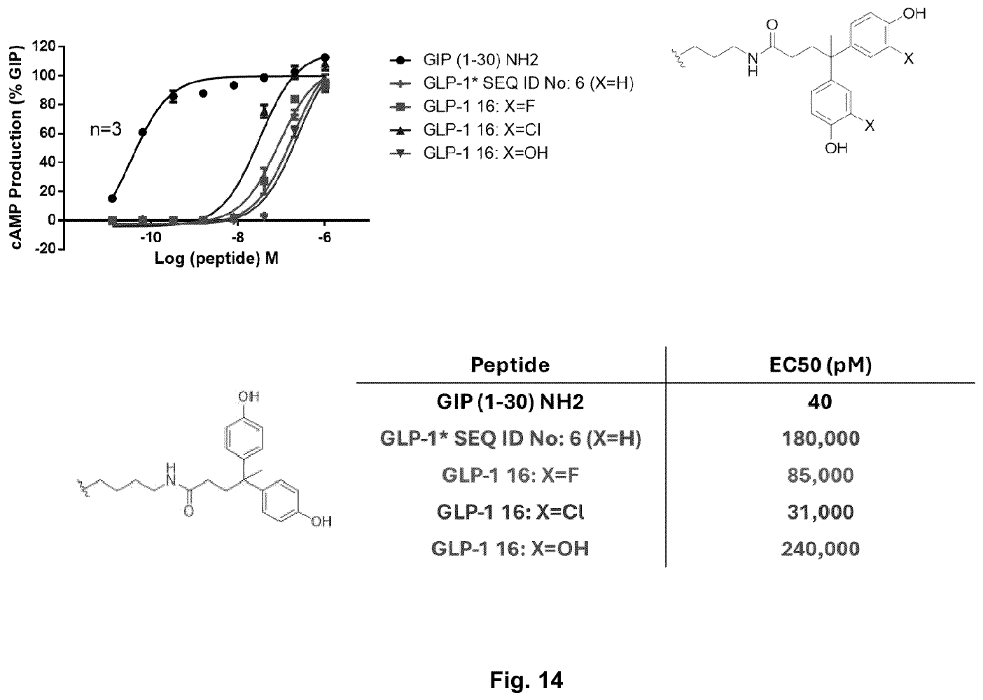

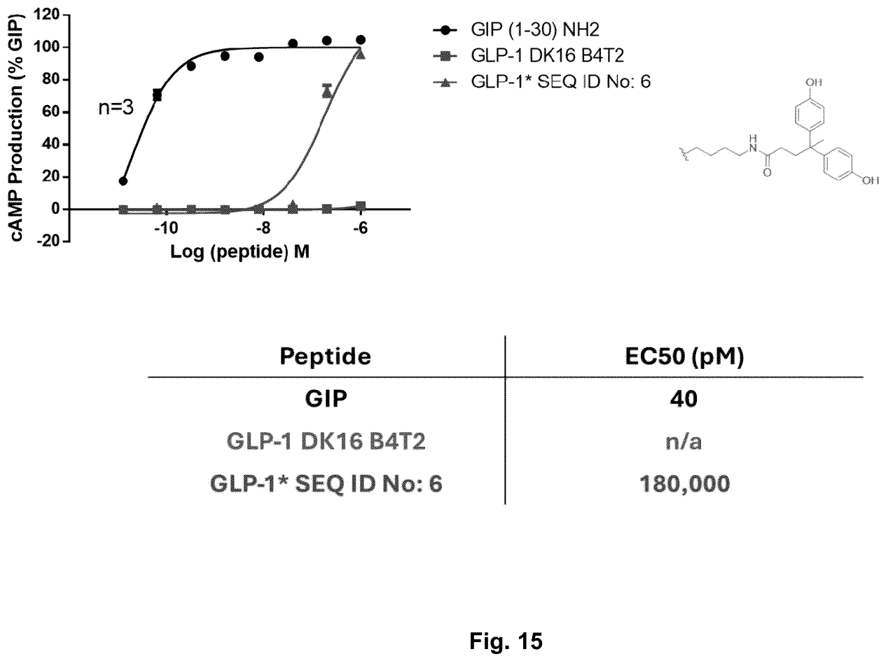

We also evaluated modifying the position ortho to the hydroxyl group in the bisphenol (FIG. 14). As shown, when a halogen is added to the ortho position, the potency of the compound increases. The addition of an additional hydroxyl group had minimal effect on the overall potency of the molecule.

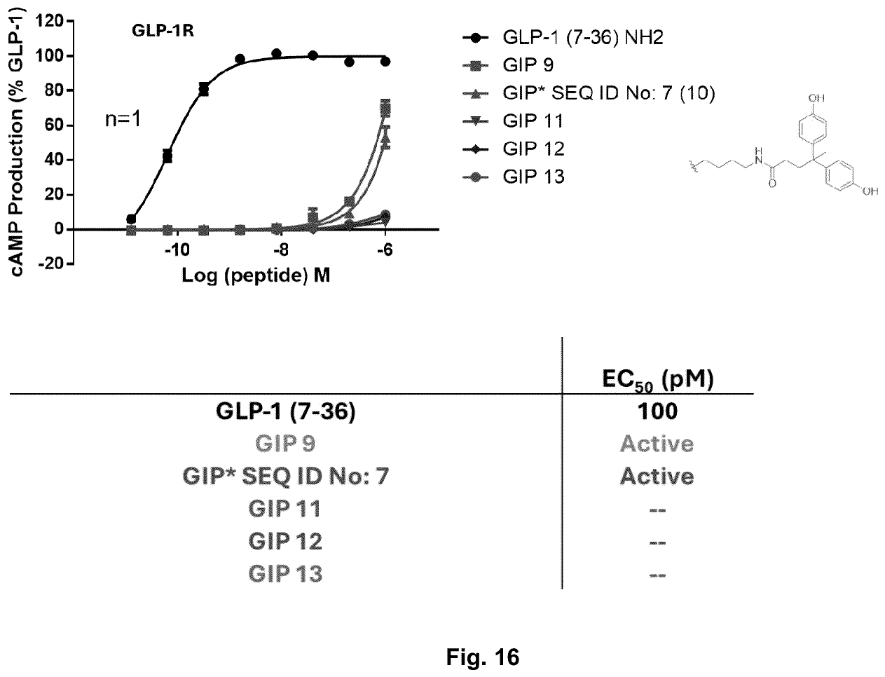

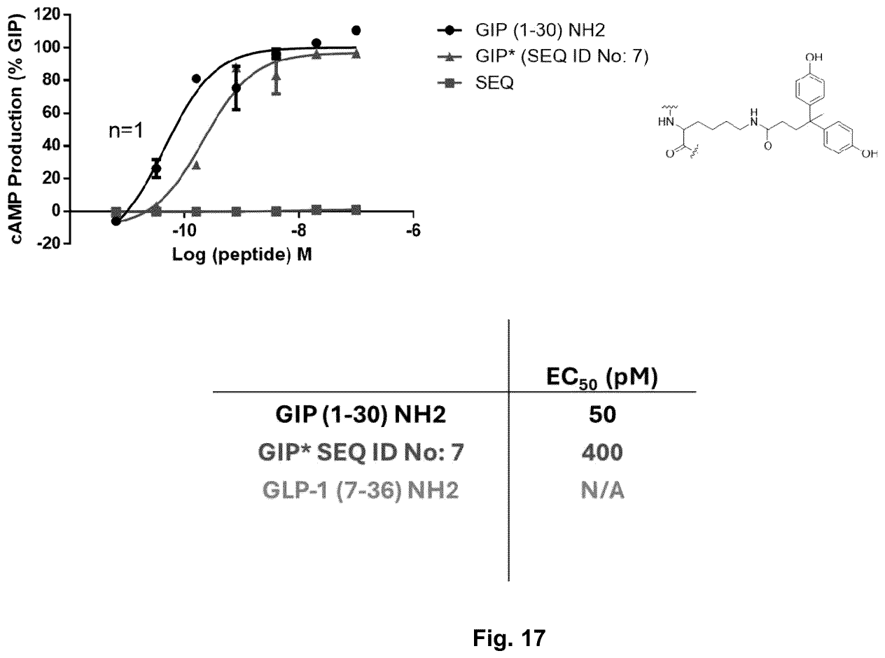

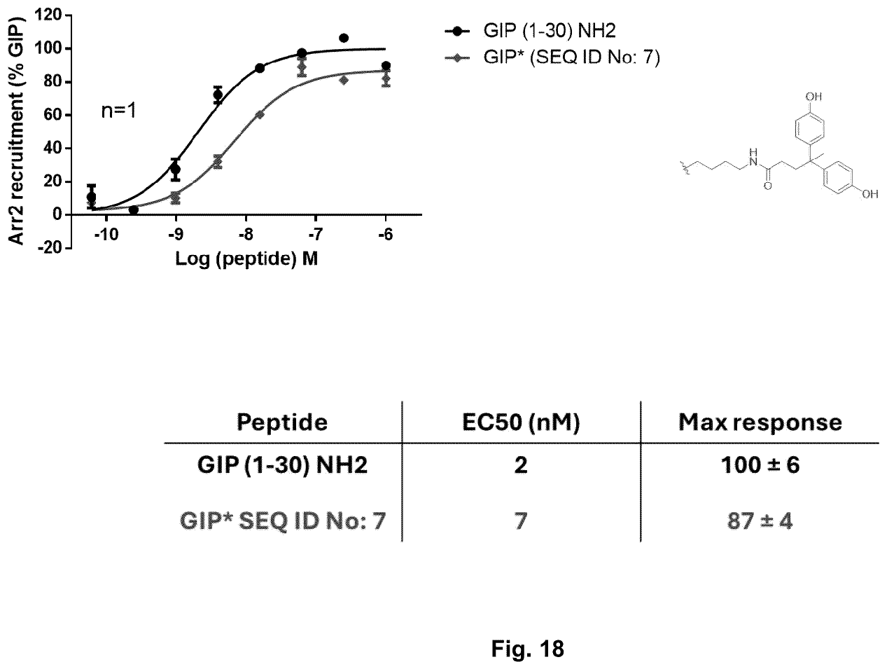

Various GIP analogues were made, and the effects of the backbone position, side chain length, amide position, and various appendages were examined. For the activation of GLP-1R, the modification at backbone positions 9 and 10 of GIP were active compared to the modifications at other positions (FIG. 16). The GIP analogue of interest at the GLP-1R (SEQ ID NO: 7) had potency similar to that of GIP itself at the GIPR, in terms of stimulating cAMP production or recruiting β-arrestin-2. (FIG. 17-18).

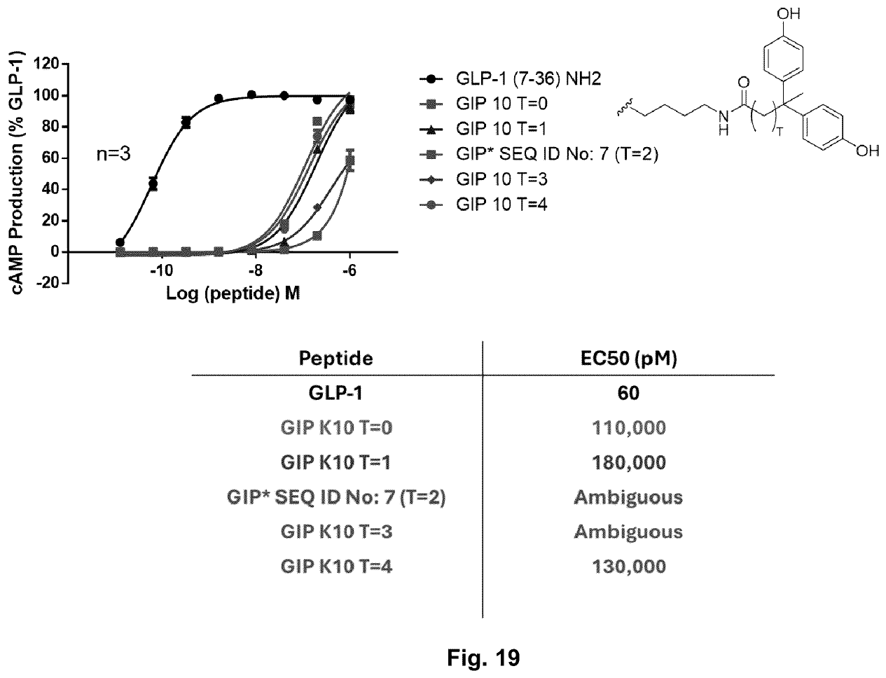

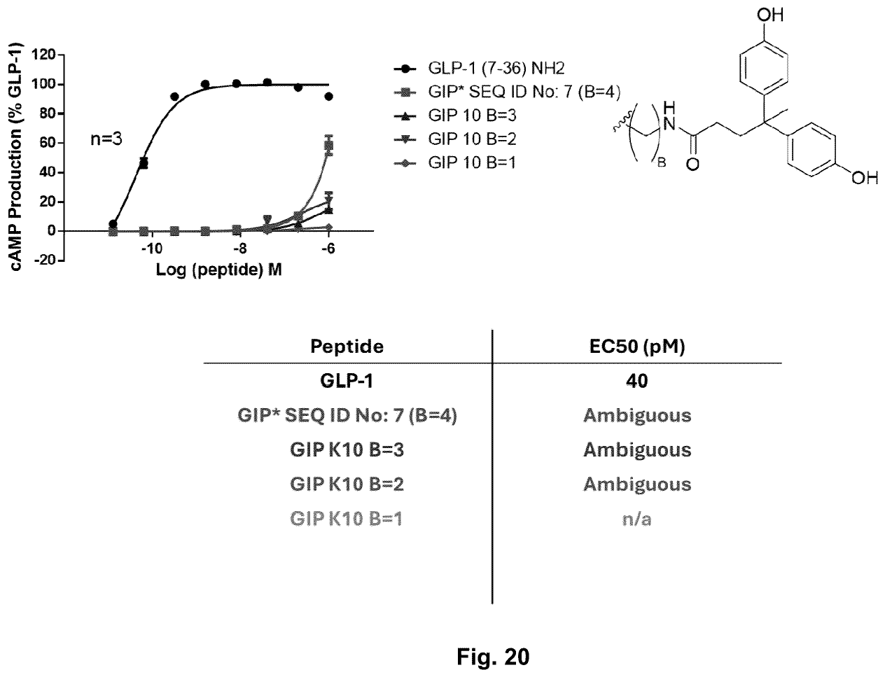

The effects of the side chain length of the modification at backbone position 10 of GIP were examined. For the activation of GLP-1R, side chains with T=0, 1, and 4 all had similar activity (FIG. 19) Shortening the side chain from B4T2 to BIT2 significantly decreased the activity (FIG. 20).

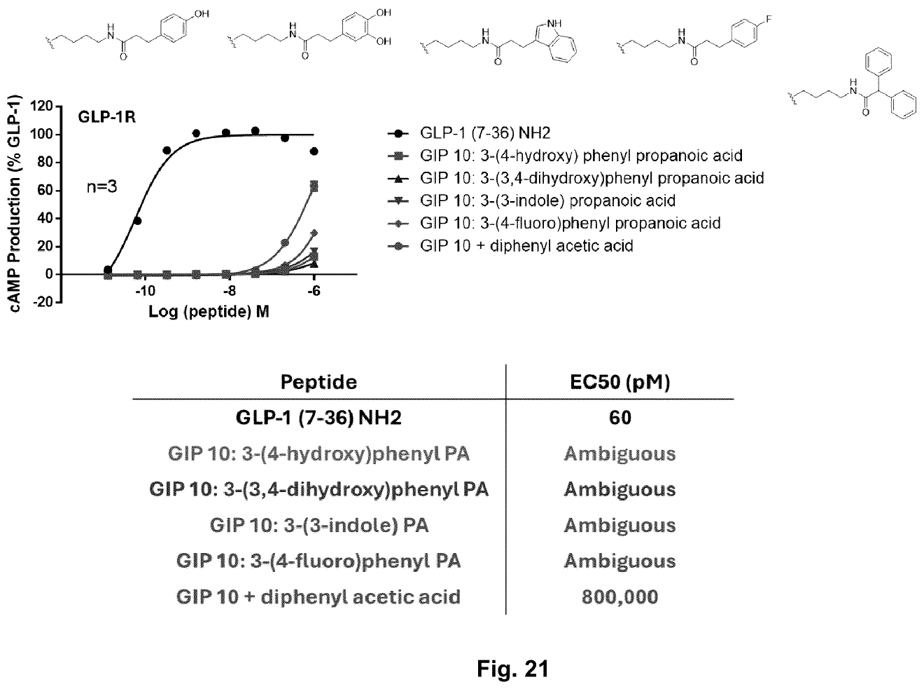

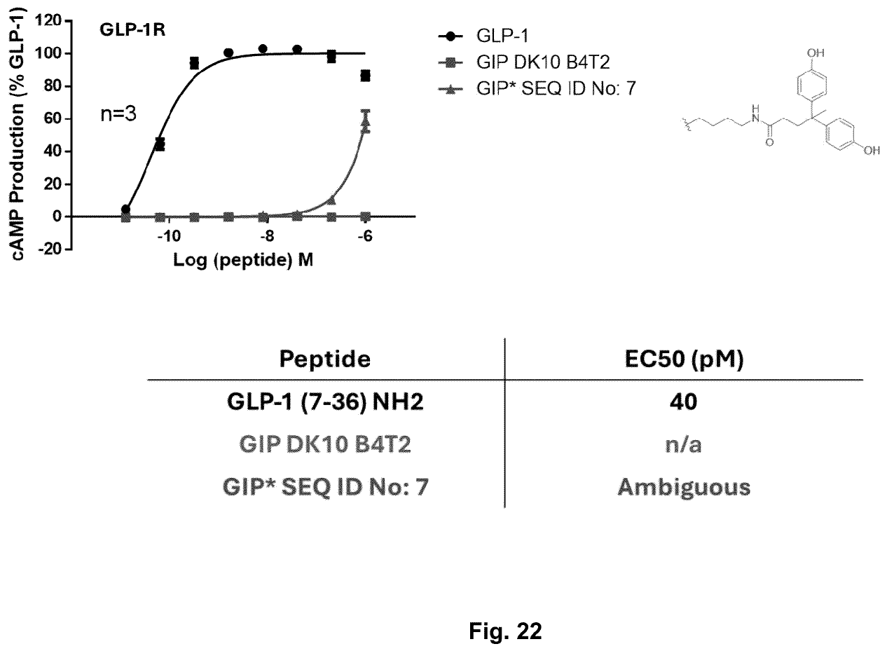

Various appendages were also examined for the GIP analogues. Overall, the bisphenol appendage exhibited greater activity than other tested appendages (FIGS. 21-22).

### Materials and Methods

| | | CATALOG # / |
|---|---|---|
| REAGENT / RESOURCE | SOURCE | IDENTIFIER |
| Peptide Synthesis | | |
| α-N-Fmoc-L-amino acids | Chem-Impex Int'l, Inc. / | Various |
| | AAPPTec (Wood Dale, Illinois, | |
| | USA) | |
| Acetonitrile HPLC-grade | Millipore Sigma (Burlington, | 34851 |
| | Massachusetts, USA) | |
| Ethyl (hydroxyimino) cyanoacetate | Chem Impex | 26426 |
| (Oxyma) | | |
| N,N′-Diisopropylcarbodiimide | Chem Impex | 00110 |
| (DIC) | | |
| Piperidine ReagentPlus ® | Millipore Sigma | 104094 |
| α-Cyano-4-hydroxycinnamic acid | Millipore Sigma | 70990 |
| (CHCA) | | |
| SPPS reaction vessel syringes/caps | Torviq (Tucson, Arizona, USA) | SF-1000/PC-SF |
| N,N Dimethylformamide (DMF) | Millipore Sigma | 494488 |
| biotech-grade | | |
| N,N Dimethylformamide (DMF) | Millipore Sigma | 319937 |
| ACS-grade | | |
| Trifluoroacetic acid (TFA) | Millipore Sigma | T6508 |
| ReagentPlus ® | | |
| 1,2 ethanedithiol | Millipore Sigma | 02390 |
| Thioanisole ReagentPlus ® | Millipore Sigma | T28002 |
| Triisopropylsilane (TIPS) | Millipore Sigma | 233781 |
| Tetrakis(triphenylphosphine)palladium(0) | Millipore Sigma | 697265 |
| Phenylsilane | Millipore Sigma | 335150 |
| Dichloroethane (DCE) | Millipore Sigma | 319929 |
| Cell Assays | | |
| Fetal Bovine Serum (FBS) | Gibco/Corning, A subsidiary of | 10082147/45000-734 |
| | Thermo Fisher Scientific | |
| | (Waltham, Massachusetts, USA) | |
| Dulbecco's Phosphate Buffered | Millipore Sigma | D8537 |
| Saline (DPBS) | | |
| HyClone ™ Penicillin-Streptomycin | GE Healthcare Life Sciences | SV30010 |
| Solution (100X) | (Wauwatosa, Wisconsin USA) | |
| Non-treated clear 96-well plates | CellTreat Scientific Products | 229590 |
| (round bottom) | Pepperell, Massachusetts, USA | |
| Nunc non-treated black 96-well | Fisher Scientific (Waltham, | 1256609 |
| plates (flat bottom) | Massachusetts, USA) | |
| Nunc non-treated T75 suspension | Fisher Scientific | 1256685 |
| flasks | | |
| Costar 50 mL Sterile Reagent | DOT Scientific Inc. (Burton, | 4870 |
| Reservoirs | Michigan) | |
| Software and Illustrations | | |
| GraphPad Prism 8 | GraphPad Software, LLC | N/A |
| | (San Diego, California, USA) | |
| Microsoft PowerPoint | Microsoft Corporation | N/A |
| | (Redmond, Washington, USA) | |
| ChemDraw Prime 16.0 | PerkinElmer (now Revvity, Inc.) | N/A |
| | (Waltham, Massachusetts, USA) | |
| Biorender | Biorender, LLC | N/A |
| | (Toronto, Ontario, Canada) | |

### Solid-Phase Peptide Synthesis:

Peptides were synthesized using a CEM Liberty Blue-brand microwave peptide synthesizer (CEM Corporation, Matthews, North Carolina, USA) at 50 μmol scale using NovaPEG Rink amide resin (Millipore-Sigma). Alloc-protected lysine was placed at the position of interest. Amino acid solutions were made in 4 molar excess of the synthesis scale. Couplings were done by adding IM DIC and 0.5 M OXYMA of appropriate amounts to the amino acid solution, stirring the solution, and adding them to the resin syringe. The coupling was done at 70° C. for 10-20 minutes. Histidine residues were done at 50° C. for 20 minutes to avoid isomerization. Deprotection of FMOC groups on amine termini was done with a 20% piperidine solution in DMF with IM OXYMA. Deprotection methods were performed at 80° C. for 2 minutes. All positions were double-coupled according to standard methods from the unit manufacturer CEM.

Bisphenol derivatives were synthesized according to previous methods. Briefly, phenol (4 eq) was dissolved in water/dioxane (0.1 M). Keto-acid (1 eq) was then added and the mixture stirred until homogeneous. H₂SO₄ (3.7 eq) was then added drop-wise and the mixture was stirred for 24-36 hours. The reaction mixture was diluted with water and extracted with EtOAc. The organic layer was then extracted exhaustively with saturated. NaHCO₃. The aqueous layer was acidified with 1 M HCl and extracted with diethyl ether. The organic layer was dried over Na₂SO₄. The product was purified via flash column chromatography using EtOAc/Pentanes and concentrated to yield the product as a yellow oil.

Isopeptide bonds were made using Alloc-protected lysine sidechains. Alloc deprotection was selectively performed using a mixture of 70 μL of phenylsilane and 14 mg of tetrakis triphenylphosphine palladium in 1 mL of DCE. The mixture was added to the syringe and placed in a MARS 6 CEM-brand microwave for 3 minutes at 35° C. This method was repeated once more to ensure full deprotection. The side chain was attached using DIC/OXYMA at the same concentrations described above. An exemplary synthesis process is shown in the following scheme:

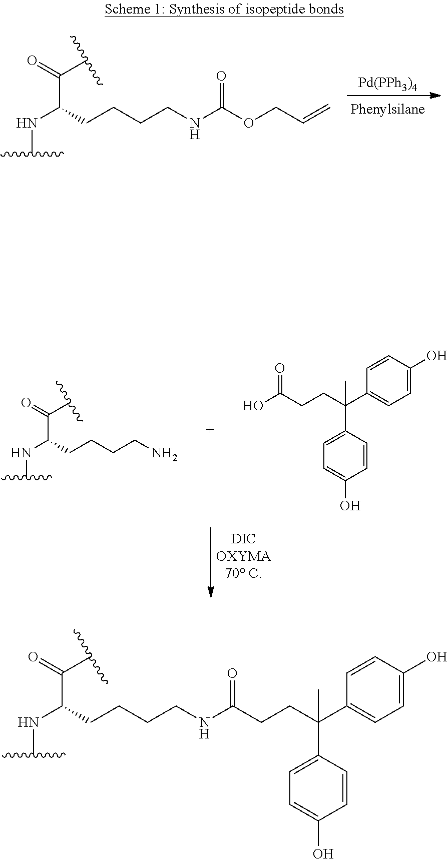 [SMILES: *N[C@@H](CCCCNC(=O)OCC=C)C(*)=O ; *N[C@@H](CCCCN)C(*)=O ; CC(CCC(=O)O)(c1ccc(O)cc1)c1ccc(O)cc1 ; *N[C@@H](CCCCNC(=O)CCC(C)(c1ccc(O)cc1)c1ccc(O)cc1)C(*)=O ; "Pd(PPh3)4"]

Cleavage of peptides from resin was done with a mixture of 92.5% trifluoroacetic acid, 5% thioanisole, and 2.5% ethanedithiol and left to sit at room temperature for 4 hours. Afterward, the solution was drained into a falcon tube and the resin washed with TFA twice. The peptide was then precipitated with 35 mL of cold diethyl ether, spun down on centrifuge, and the supernatant decanted. The peptide pellets were blown down under nitrogen and stored at −20° C. until purification.

Purification. Peptides were purified on a preparative Reverse Phase HPLC using a gradient of 25 to 50% acetonitrile in water over 25 minutes. The peaks eluted at approximately 16-22 minutes on the column and fractions collected. The fractions were compiled and acetonitrile evaporated under nitrogen. The remaining solution was frozen with dry ice and lyophilized to eliminate the remaining water. The dry peptide was re-dissolved in 1 mL of a mixture of water:ACN and the concentration tested using a NanoDrop UV/VIS spectrometer.

The peptides were then re-lyophilized and the dry peptide dissolved in the appropriate amount of DMSO to yield a 1 mM stock solution.

### Cell Culture:

HEK293-GS22 cells (GloSensor-brand CAMP Assay, Promega Corporation, Fitchburg, Wisconsin, USA, catalog nos. E1171, E1290, E1291, and E2301) were cultured in 75 cm², culture-treated, vented flasks (Corning) at 37° C. with 5% CO₂. Cell medium was 0.22 μm-filtered DMEM supplemented with 10% (v/v) FBS (referred to as GS media) and penicillin/streptomycin.

Cells were passaged every 4-5 days at confluency. For passaging, cells were washed with 6 mL of DPBS and treated with 3 mL of 0.05% EDTA/trypsin. 6 mL of media was added to the flask and the solution transferred to a falcon tube where it was spun down for 5 minutes at 800 rpm and the supernatant removed. The cells were resuspended in 6 mL of media and aliquots placed in 12 mL of media at a dilution of either 1:30 or 1:40 depending on growth rate.

Generating HEK cells stably expressing GIPR. Low passage HEK 293 cells stably expressing Glosensor-22F (Promega Corporation, Madison, Wisconsin, USA) luminescent CAMP-sensing protein (HEK 293 GS cells) were transfected using 10 ug GIPR-Flag with 30 uL linear polyethyleneimine (PEI) 10 kDa in 1 mL of DMEM. The next day the cells were split 1:3 in a 10 cm plate. Selection was performed the following day using 100 ug/mL Zeocin for two weeks to allow cells expressing the receptor to outcompete the wildtype cells. cAMP measurements and proteomics (discussed below) of purified receptor confirmed successful expression. Cells were maintained using the typical media (10% FBS in DMEM) supplemented with 50 μg/mL Zeocin in place of penicillin-streptomycin.

### CAMP Assay:

The cells were passaged and resuspended in 11 mL of 10% FBS in DMEM and 100 μL was placed in each well of an appropriately treated adherent white 96-well plate. This was left to sit in the incubator overnight.

On the day of the assay, the plate was removed from the incubator and the media removed from the 96-well plate. A solution was made by combining an aliquot of D-luciferin and 11 mL of DPBS. 90 μL of this was placed in each well and incubated for 20 minutes at room temperature in the dark so that the luciferin was not deactivated. Meanwhile, peptide stocks were made in a separate dilution 96-well plate by serially diluting the 1 mM peptide stocks. 1 μL of stock solution was placed in 1 mL of DPBS and 200 μL of this mixture was placed in the top well of the plate. 200 μL of DPBS was placed in each well below this. Then, 50 μL was serially diluted down the plate to yield the concentrations of peptide for the assay. 10 μL from each well of this dilution plate was added to the cell assay plate and immediately taken to the plate reader for 30 minutes of luminescence readings. Data is analyzed from the read with the highest values.
Arrestin Recruitment Assay:

On day 1 of assay preparation, HEK293 GS22 cells were passaged and placed in appropriately treated adherent cell culture dishes at a 1:3 dilution rate in a media of 10% FBS in DMEM. They were grown overnight at 37° C. with 5% CO₂. On day 2, the media was removed and replaced with 4.5 mL of raw DMEM and incubated for 20 minutes. On day 3, the cells were passaged and plated following the procedure described above for the CAMP assay.

On the day of the assay, the cells in the plate were washed with DPBS+glucose two times by removing 85 μL of liquid from each well of the plate, replacing with DPBS+glucose, and repeating one more time. The second wash solution was left in the incubator for 45 minutes to 1 hour. Meanwhile, peptide dilutions were made by taking 2 μL of 1 mM peptide stock in DMSO and diluting it in 200 μL of PBS. 100 μL of this dilution was placed in the top well of a dilution plate and 90 μL of PBS was placed in each of the remaining wells. 30 μL was diluted down the plate to yield the final dilution stocks. 10 μL aliquots of these dilutions were added to the cell plate and the plate incubated at room temperature for 20 minutes. Meanwhile, the substrate stock was made by diluting 60 μL of Deep Blue C (colenterazine) in 850 μL of DPBS and 420 μL of EtOH. 10 μL of this substrate was added to the cell plate and immediately placed on the plate reader with emission cartridges reading out at 528 nm and 400 nm. Data was taken from between 15 and 45 minutes of the plate reader method for data analysis.

### Data Analysis:

Data analysis was performed using GraphPad Prism version 6 software with the Log Agonist+3 parameters method to generate EC₅₀ curves. The data were normalized so that native GLP-1 peptide was 100% and vehicle (stock made with 1 μL of DMSO instead of peptide stocks) was 0%.

SEQUENCE LIST
GLP-1 (7-37):
HAEGTFTSDV SSYLEGQAAK EFIAWLVKGR G (SEQ ID NO: 1)
GLP-1 (7-36)-NH₂:
HAEGTFTSDV SSYLEGQAAK EFIAWLVKGR-NH₂ (SEQ ID NO: 2)
GIP (1-42):
YAEGTFISDY SIAMDKIHQQ DEVNWLLAQK GKKNDWKHNI TQ (SEQ ID NO: 3)
GIP (1-30)-NH₂:
YAEGTFISDY SIAMDKIHQQ DEVNWLLAQK-NH₂ (SEQ ID NO: 4)
Tirzepatide:
YUEGTGTSDY SIULDKIAOK* AFVOWLIAGG PSSGAPPS-NH₂ (SEQ ID NO: 5)
Single Underline = a residue present in GLP-1 only
Double Underline = a residue present in GIP only
Bold, Italic = a residue present in both GLP-1 and GIP
Standard Font = a residue not present in either GLP-1 or GIP
K* = Lys bearing a fatty acid-like appendage
Single Site-Modified GLP-1 (“GLP-1*”):
HAEGTFTSDK* SSYLEGQAAK EFIAWLVKGR G-NH₂ (SEQ ID NO: 6)
K* = Lys having a modified sidechain:

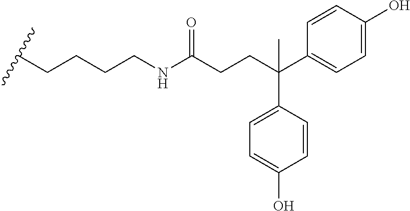 [SMILES: *CCCCNC(=O)CCC(C)(c1ccc(O)cc1)c1ccc(O)cc1]

Single Site-Modified GIP (GIP*):
YAEGTFISDK* SIAMDKIHQQ DEVNWLLAQK-NH₂ (SEQ ID NO: 7)
K* = Lys having a modified sidechain:

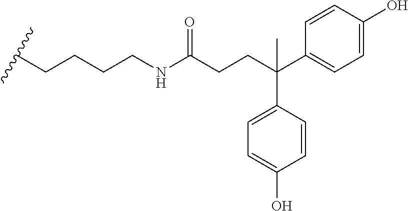 [SMILES: *CCCCNC(=O)CCC(C)(c1ccc(O)cc1)c1ccc(O)cc1]

What is claimed is:

1. A method of making dual GLP-1 receptor and GIP receptor agonists, the method comprising modifying at least one side chain of a residue in GLP-1 and/or GIP, to yield a modified GLP-1 and/or GIP, wherein the modified GLP-1 and GIP are agonists of both GLP-1 receptor and GIP receptor.

2. The method of claim 1, wherein the at least one side chain of a residue in GLP-1 and/or GIP is modified to contain a side chain comprising: 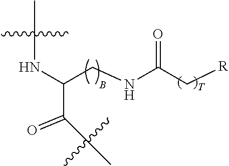
wherein:
B is an integer of from 1 to 6;
T is an integer of from 0 to 6, or the carbon atom bearing the subscript T is a substituted or unsubstituted C₃-C₆-cycloalkyl and T=1; and
R is selected from the group consisting of polycyclic aromatic hydrocarbons and 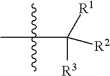
wherein R¹ is independently selected from the group consisting of an unsubstituted polycyclic aromatic hydrocarbon, a polycyclic aromatic hydrocarbon having up to three substitutions, an unsubstituted heterocycle, a heterocycle having up to three substitutions, a halogen, and 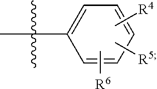
wherein R⁴, R⁵, and R⁶ are independently selected from the group consisting of H, halogen, —OH, —C₁-C₆-linear or branched, unsubstituted or alkyl, and —O—C₁-C₆-linear or branched, substituted or unsubstituted alkyl; and
R² and R³ are independently selected from the group consisting of H, halogen, —OH, —C₁-C₆-linear or branched, substituted or unsubstituted alkyl, —C₁-C₆-linear or branched, substituted or unsubstituted alkylaryl, —O—C₁-C₆-linear or branched, substituted or unsubstituted alkyl, —O—C₁-C₆-linear or branched, substituted or unsubstituted alkylaryl, and the groups listed for R¹.

3. The method of claim 1, consisting essentially of modifying one (1) and only (1) side chain of a residue in the GLP-1 and/or GIP.

4. The method of claim 2, wherein R is

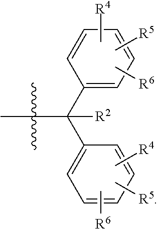

5. The method of claim 4, wherein R² R⁴, R⁵, and R⁶ are independently selected from the group consisting of a halogen, H, —OH, —CH₃, and —O—CH₃.

6. The method of claim 5, wherein “T” and “B” are integers of from 1 to 4, R¹, R⁴, R⁵, and R⁶ are selected from the group consisting of —OH or —O—CH₃, and R² is H or —CH₃.

7. The method of claim 1, wherein the carbon atom bearing the subscript T is cyclohexyl and T=1.

8. The method of claim 1, wherein at least one other side chain of a residue in GLP-1 and/or GIP is modified to comprise a side chain selected from the group consisting of:

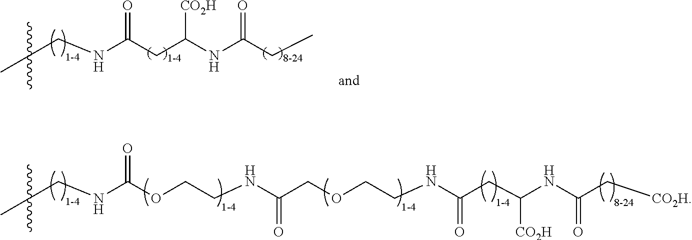

9. A compound comprising GLP-1 or GIP in which at least one side chain of a residue in the GLP-1 and/or the GIP is modified to contain a side chain comprising: 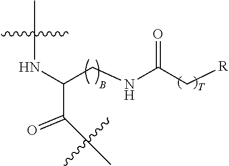
wherein:
B is an integer of from 1 to 6;
T is an integer of from 0 to 6, or the carbon atom bearing the subscript T is a substituted or unsubstituted C₃-C₆-cycloalkyl and T=1; and
R is selected from the group consisting of polycyclic aromatic hydrocarbons and 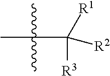
wherein R¹ is independently selected from the group consisting of an unsubstituted polycyclic aromatic hydrocarbon, halogen, a polycyclic aromatic hydrocarbon having up to three substitutions, an unsubstituted heterocycle, a heterocycle having up to three substitutions, and 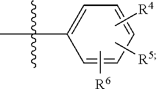
wherein R⁴, R⁵, and R⁶ are independently selected from the group consisting of H, halogen, —OH, —C₁-C₆-linear or branched, unsubstituted or alkyl, and —O—C₁-C₆-linear or branched, substituted or unsubstituted alkyl; and
R² and R³ are independently selected from the group consisting of H, halogen, —OH, —C₁-C₆-linear or branched, substituted or unsubstituted alkyl, —C₁-C₆-linear or branched, substituted or unsubstituted alkylaryl, —O—C₁-C₆-linear or branched, substituted or unsubstituted alkyl, —O—C₁-C₆-linear or branched, substituted or unsubstituted alkylaryl, and the groups listed for R¹;
and salts thereof.

10. The compound of claim 9, wherein one (1) and only (1) side chain of a residue in the GLP-1 and/or GIP is modified.

11. The compound of claim 9, wherein R is

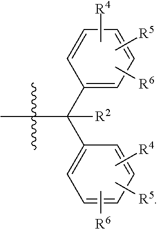

12. The compound of claim 11, wherein R², R⁴, R⁵, and R⁶ are independently selected from the group consisting of a halogen, H, halogen, —OH, —CH₃, and —O—CH₃.

13. The compound of claim 12, wherein “T” and “B” are integers of from 1 to 4, R⁴ R⁵, and R⁶ are independently selected form the group consisting of —OH and —O—CH₃, and R² is H or —CH₃.

14. The compound of claim 9, wherein the carbon atom bearing the subscript T is cyclohexyl and T=1.

15. The compound of claim 9, wherein the salt is a pharmaceutically suitable salt.

16. A compound according to claim 9, wherein at least one other side chain of a residue in GLP-1 and/or GIP comprises a side chain selected from the group consisting of:

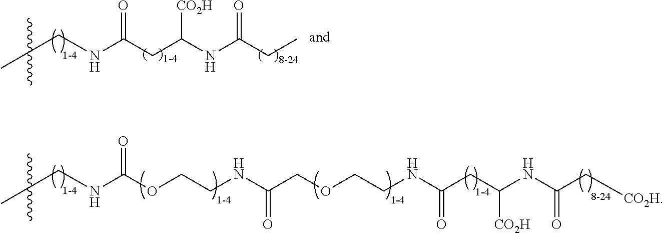

17. A pharmaceutical composition comprising a compound as recited in claim 9, optionally in combination with a pharmaceutically suitable carrier.

18. A method of treating diabetes and/or obesity in a mammal, the method comprising administering an anti-diabetes-effective amount and/or a weight loss-effective amount of a compound as recited in claim 9 to a mammal.

19. The method of claim 18, comprising administering the compound to a human being.
